(19) United States
(12) Patent Application Publication
(10) Pub. No.: US 2023/0263479 A1
(43) Pub. Date: Aug. 24, 2023

(54) SENSING SYSTEMS AND METHODS FOR PROVIDING DIABETES DECISION SUPPORT USING CONTINUOUSLY MONITORED ANALYTE DATA

(71) Applicant: Dexcom, Inc., San Diego, CA (US)

(72) Inventors: Matthew Lawrence JOHNSON, San Diego, CA (US); Samuel Isaac EPSTEIN, San Diego, CA (US); Sarah Kate PICKUS, San Diego, CA (US); Lauren Hruby JEPSON, San Diego, CA (US); Kevin CHENG, San Diego, CA (US); Spencer Troy FRANK, San Diego, CA (US); Qi AN, San Diego, CA (US); Devon M. HEADEN, San Diego, CA (US); Abdulrahman JBAILY, San Diego, CA (US)

(21) Appl. No.: 18/052,194

(22) Filed: Nov. 2, 2022

Related U.S. Application Data
(60) Provisional application No. 63/263,540, filed on Nov. 4, 2021.

(51) Int. Cl. A61B 5/00; A61B 5/11; G16H 20/00; A61B 5/155; A61B 5/145

(52) U.S. Cl. CPC A61B 5/7275; A61B 5/1118; G16H 20/00; A61B 5/7203; A61B 5/155; A61B 5/14532

(57) ABSTRACT

Certain aspects of the present disclosure relate to methods and systems for predicting glycemic events in a patient induced as a result of physical activity. In certain aspects, a method includes monitoring a plurality of analytes of the patient continuously during a time period to obtain analyte data, the plurality of analytes including at least glucose and lactate. The method further includes processing the analyte data from the time period to determine an intensity level of physical activity engaged by the patient during the time period. The method further includes generating a glycemic event prediction using at least the analyte data for the plurality of analytes and the determination of physical activity intensity. The method further includes generating one or more recommendations for treatment for the patient based, at least in part, on the glycemic event prediction.

### CROSS-REFERENCE TO RELATED APPLICATION(S)

This application claims benefit of and priority to U.S. Provisional Patent Application No. 63/263,540, filed Nov. 4, 2021. The aforementioned application is herein incorporated by reference in its entirety.

### BACKGROUND

Diabetes mellitus is a metabolic condition in which the pancreas cannot create sufficient insulin (Type I or insulin dependent) and/or in which insulin has reduced efficacy (Type 2 or non-insulin dependent). Insulin is a hormone that allows the body to use glucose for energy, or store glucose as fat. In the diabetic state, the patient suffers from high glucose, called “hyperglycemia,” which may cause an array of negative physiological effects (for example, nerve damage (neuropathy), kidney failure, skin ulcers, diabetic ketoacidosis, or bleeding into the vitreous of the eye) associated with the deterioration of small blood vessels. Conversely, the state of having low blood glucose is called “hypoglycemia.” Severe hypoglycemia can lead to damage of the heart muscle, neurocognitive dysfunction, and in certain cases, seizures or even death.

In light of the above, it is extremely important for patients with diabetes to be constantly aware of their glycemic state, so as to know if and what steps they should take to manage their diabetic condition, in order to stay within a target glucose range and avoid physical complications. Thus, diabetic patients can benefit from continuous monitoring of their glucose levels and trends, in conjunction with real-time diabetes management guidance that is determined based on the monitored data. However, management of diabetes still presents many challenges for patients, clinicians, and caregivers, as a confluence of various factors impacts a patient's glycemic state, and these factors may not always be reflected in the patient's glucose levels and glucose trends, thereby affecting the accuracy of diabetes management guidance provided.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIGS. 4A-4B illustrate a flow diagram of an example method for providing decision support using a continuous analyte monitoring system configured to continuously measure at least glucose and lactate levels, in accordance with some example aspects of the present disclosure.

FIG. 5 is a flow diagram depicting a method for training machine learning models to provide a prediction of physical activity-induced glycemic events, according to certain embodiments of the present disclosure.

FIG. 6 is a block diagram depicting a computing device configured to perform the operations of FIGS. 4A-5, according to certain embodiments disclosed herein.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Management of diabetes presents many challenges for patients, clinicians, and caregivers, as a confluence of various factors impacts a patient's glycemic state, and these factors may not always be reflected in a patient's glucose levels and glucose trends.

Among other things, physical activity (e.g., exercise, training, outdoor activities, sporting events, and other events involving physical exertion of a patient) presents particularly complex challenges in determining diabetes management guidance for patients with diabetes. For example, physical activity may induce hyper- or hypoglycemic events in patients which may require immediate medical intervention. However, glucose levels and trends alone do not provide a comprehensive view of a patient's physiological state during physical activity, and may in certain instances resemble levels and trends of, e.g., a nutrition-related event rather than physical activity of the patient. As a result, basing diabetes management guidance solely on the glucose levels and/or glucose trends of a patient during a physical activity may result in inaccurate or incorrect guidance being provided.

Because regular physical activity, in combination with diet and medication treatments, is crucial for disease management of patients with metabolic disorders such as diabetes, there is a need in the art for improved systems and methods for characterizing a patient's physiological state during physical activity for improved diabetes management support, such as providing more accurate prediction of hyper- and hypoglycemia, etc.

In light of the above, patients with diabetes can benefit from real-time diabetes management guidance that is determined based on a physiological state of the patient. Conventionally, the physiological state of the patient is determined based on glucose levels and glucose trends, which then inform the prediction of hyper- and/or hypoglycemic events (hereinafter “adverse glycemic events” or “glycemic events”) and the type of guidance provided to the patient. However, management of diabetes presents many challenges for patients, clinicians, and caregivers, as a confluence of various factors can impact a patient's glucose level and glucose trends, thus affecting the accuracy of guidance provided.

The present inventors have recognized, among other things, that physical activity, e.g., workouts, training, sporting events, and other events involving physical exertion of a patient, present particularly complex challenges in determining a physiological state of a patient when utilizing only glucose levels and glucose trends, and thus, make it challenging to provide accurate guidance for managing the patient's diabetes. For example, it is known that physical activity may induce adverse glycemic events in patients which require medical intervention. However, glucose levels and trends alone do not provide a comprehensive view of a patient's physiological state during such physical activity. For example, in some instances, other confounding factors may contribute to the observed glucose levels and trends. In one example, the observed levels and trends may in certain instances resemble levels and trends of, e.g., a nutrition-related event rather than physical activity by the patient. Thus, a diagnostic system utilizing only glucose levels and trends may incorrectly characterize the physiological state of the patient during physical activity, leading to inaccurate prediction of adverse glycemic events resulting from the physical activity and poor guidance on how to manage such event, which may result in deterioration of a patient's condition.

To better characterize the physiological state of a patient participating in a physical activity, certain available diagnostics systems utilize additional data from various types of physical activity sensors such as heart rate monitors and accelerometers. However, these physical activity sensors are more accurate in suggesting physical stress (i.e., intensity) levels for certain types of physical activity, e.g., cardiovascular activities such as running, as compared to others, such as weightlifting, yoga, etc. Thus, data provided from such physical activity sensors may not paint a complete picture of a patient's physical stress levels during certain types of activities, leading to suboptimal or inaccurate characterization of the patient's physiological state.

Because regular physical activity, in combination with diet and medication treatments, is crucial for disease management of patients with metabolic disorders such as diabetes, there is a need in the art for improved systems and methods for characterizing a patient's physiological state during physical activity for improved diabetes management support, such as providing more accurate prediction of physical activity-induced adverse glycemic events, etc.

Accordingly, certain embodiments described herein provide a technical solution to the technical problem described above by providing a continuous analyte monitoring system that is configured to generate and analyze a combination of, at least, interstitial glucose and lactate measurements obtained from one or more continuous analyte sensors, in order to provide more accurate predictions of current or future physical activity-induced glycemic events for patients with diabetes, as well as decision support for managing diabetes of patients as related to physical activity, e.g., exercise. Decision support may include risk assessment, diagnosis, and/or recommendations for treatment of adverse glycemic events induced as a result of physical activity, or for overnight adverse glycemic events.

Continuously monitoring at least a combination of glucose and lactate levels enables a better characterization of the physiological state of a patient, and in particular, a patient participating in a physical activity. The major function of glucose is to provide energy for cellular functions. Glucose is broken down during the process of cellular respiration into various byproducts, and along the way, an energy source called adenosine 5′-triphosphate (ATP) is produced. ATP is the principal molecule for storing and transferring energy in cells.

Depending on the presence of oxygen, glucose may be broken down via aerobic cellular respiration (in the presence of oxygen) or anaerobic cellular respiration (in the absence of oxygen). During aerobic cellular respiration, glucose is first converted to pyruvate in a process called glycolysis, releasing some initial ATP. Pyruvate is then converted to acetyl CoA, which is processed through the citric acid cycle to release additional ATP. Electron carriers from these reactions transfer their electrons into a series of protein complexes in the inner membrane of the mitochondrion called the electron transport chain, and the flow of these electrons through the electron transport chain drives synthesis of additional ATP.

During anaerobic cellular respiration, glucose is first converted to pyruvate via anaerobic glycolysis. Pyruvate is then converted to lactate, a conjugate base of lactic acid, by the enzyme lactate dehydrogenase (LDHA). The produced lactate is then used by surrounding tissues as fuel, or is transported to other tissues for use as an energy source. This process, though producing less energy than the full energy potential of a glucose molecule, is very rapid and provides an immediate energy source.

Aerobic cellular respiration occurs during normal metabolism and low to moderate level physical activity, when oxygen is readily available to be utilized in the breakdown of glucose for energy. Anaerobic cellular respiration, however, occurs during elevated metabolism and moderate to high level physical activity, when the oxygen demand of muscles surpasses the oxygen supply. Accordingly, in certain situations, the presence of lactate may better indicate the type and intensity of physical activity engaged by a user than, e.g., glucose levels alone. Thus, continuous monitoring of lactate, in addition to glucose, may be needed to assess the physical activity engaged by a user in order to accurately predict glycemic events resulting from the user engaging in such physical activity.

In certain embodiments described herein, the continuous analyte monitoring system may provide decision support to the patient based on a variety of collected data, including analyte data, patient information, secondary sensor data (e.g., non-analyte data), patient input, etc. For example, the analyte data may include continuously monitored lactate data in addition to other continuously monitored analyte data, such as glucose, ketones, glycerol, electrolytes such as sodium and potassium, calculated measurements such as anion gap, and other suitable analytes. The continuously monitored lactate data may indicate, or be used for determining the patient's lactate levels, lactate production levels, and/or lactate clearance rates.

As described above, the collected data also includes patient information, which may include age, gender, glucose threshold levels, previous exercise-related or nutrition related event data, fitness levels, activity frequency, etc. Secondary sensor data may include accelerometer data, step rate data, exercise equipment power meter data, global positioning system (GPS) data, heart rate, heart rate reserve, heart rate variability (HRV), electrocardiogram (EKG) data, electromyogram (EMG), respiration rate, temperature, blood pressure, galvanic skin response, oxygen uptake data (e.g., VO₂ max), sleep, impedance data, etc.

According to certain embodiments of the present disclosure, the decision support system presented herein is designed to provide a prediction of a current or future physical activity-induced glycemic event for patients with diabetes, as well as diabetes decision support to assist the patient in managing their diabetes. Providing diabetes decision support may involve using large amounts of collected data, including for example, the analyte data, patient information and secondary sensor data mentioned above, to (1) automatically detect the patient engaging in physical activity; (2) determine relevant parameters of the physical activity engaged by the patient (e.g., intensity, duration, and/or type of physical activity); (3) predict a current or future glycemic event based, in part, on the physical activity engaged by the patient, and (4) make patient-specific treatment decisions or recommendations for diabetes. In other words, the decision support system presented herein may offer information to direct and help improve care for patients with diabetes.

In certain embodiments, the decision support system described herein may use various algorithms or artificial intelligence (AI) models, such as machine-learning models, trained based on patient-specific data and/or population data to provide real-time, or retrospective, decision support to a patient based on the collected information about the patient. For example, certain aspects are directed to algorithms and/or machine-learning models designed to predict a current or future physical activity-induced glycemic event of the patient based, in part, on the physical activity engaged by the patient. The algorithms and/or machine-learning models may be used in combination with one or more continuous analyte sensors configured to continuously measure at least glucose and lactate levels to provide real-time or retrospective prediction of a current or future glycemic event for the patient. In particular, the algorithms and/or machine-learning models may take into account parameters, such as normal lactate and glucose ranges of a patient (e.g., normal minimum and maximum levels), when predicting glycemic events. Based on these parameters, the algorithms and/or machine-learning models may provide a prediction of a current or future physical activity-induced glycemic event of the patient, as well as a recommendation for treatment to manage (e.g., prevent or abate) the predicted glycemic event. The algorithms and/or machine-learning models may take into consideration population data, personalized patient-specific data, or a combination of both when predicting glycemic events for the patient.

According to certain embodiments, prior to deployment, the machine learning models are trained with training data, e.g., including population data. As described in more detail herein, the population data may be provided in a form of a dataset including data records or samples of historical patients with diabetes. Each data record is then featurized (e.g., refined into a set of one or more features, or predictor variables) and labeled. Data labeling is the process of adding one or more meaningful and informative labels to provide context to the data for learning by the machine learning models. In certain embodiments, each data record is labeled with one or more of a glycemic event (e.g., including the type of glycemic event, the time associated with the glycemic event, the glucose level associated with the glycemic event, the duration of the glycemic event, etc.), a treatment provided in response to a glycemic event (e.g., including the type of treatment, the amount/dosage of the treatment, the result of the treatment, etc.), etc. The features associated with each data record may be used as input into the machine learning models, and the generated output may be compared to label(s) assigned to each of the data records. The models may compute a loss based on the difference between the generated output and the provided label(s). This loss can then be used to modify the internal parameters or weights of the models. By iteratively processing features associated with each data record corresponding to each historical patient, the models may be iteratively refined to generate accurate predictions of glycemic events of patients engaging in physical activity.

The combination of a continuous analyte monitoring system with machine learning models and/or algorithms for predicting glycemic events of diabetic patients engaging in physical activity, as provided by the decision support system described herein, enables real-time or retrospective prediction and provision of treatment decisions or recommendations to allow early intervention. In particular, the decision support system may be used to provide an early alert of hyper- or hypoglycemic events. Early detection of such events may allow for earlier intervention to prevent or abate such events from leading to further physical complications, which may lead to hospitalization and even death, in some cases.

In addition, through the combination of a continuous analyte monitoring system with machine learning models and/or algorithms for predicting glycemic events of diabetic patients engaging in physical activity, the decision support system described herein may provide the necessary accuracy and reliability that diabetic patients expect from continuous analyte monitoring systems. For example, the continuous monitoring of multiple analytes, e.g., glucose and lactate, in combination with machine learning models and/or algorithms for predicting glycemic events as affected by physical activity, may better characterize a patient's physiological state during and/or after a physical activity when compared to utilizing glucose levels and/or glucose trends alone. Accordingly, the decision support system described herein may provide more reliable predictions of glycemic events of diabetic patients engaging in physical activity.

### Example Decision Support System Including an Example Continuous Analyte Sensor for Predicting a Glycemic Event of a Patient Engaging in Physical Activity

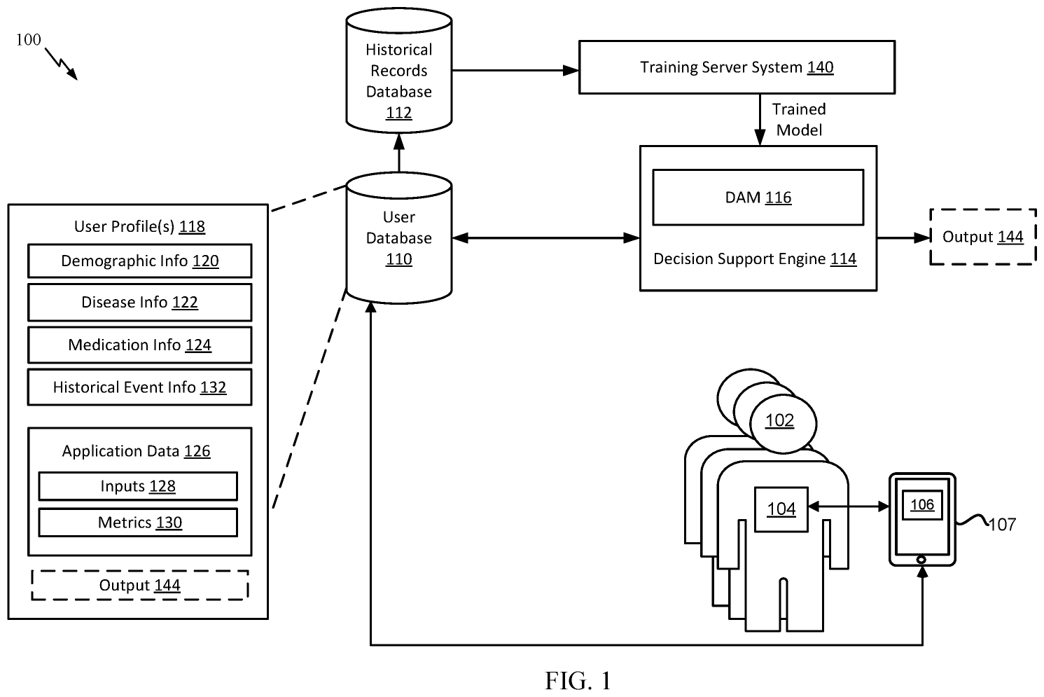

FIG. 1 illustrates an example decision support system 100 for predicting current or future physical activity-induced glycemic events of users 102 (individually referred to herein as a user and collectively referred to herein as users), using a continuous analyte monitoring system 104 configured to continuously measure at least glucose and lactate levels. A user, in certain embodiments, may be a diabetes patient or, in some cases, the patient's caregiver. In certain embodiments, system 100 includes continuous analyte monitoring system 104, a display device 107 that executes application 106, a decision support engine 114, a user database 110, a historical records database 112, a training server system 140, and an decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; potassium, quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include glucose, lactate, ketones, and in some cases other analytes listed, but not limited to, above may also be considered.

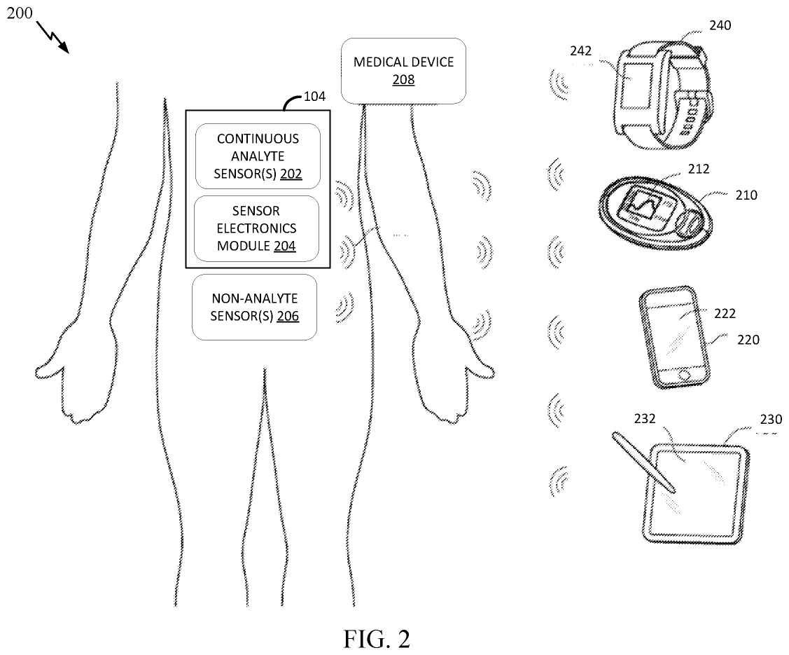

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a fitness tracker, a tablet, or any other computing device capable of executing application 106. Continuous analyte monitoring system 104 may be described in more detail with respect to FIG. 2.

Application 106 is a mobile health application that is configured to receive and analyze analyte measurements from analyte monitoring system 104. For example, application 106 stores information about a user, including the user's analyte measurements, in a user profile 118 of the user for processing and analysis as well as for use by the decision support engine 112 to provide decision support recommendations or guidance to the user. Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114, may provide decision support recommendations to the user via application 106. Decision support engine 114 provides decision support recommendations based on information included in user profile 118.

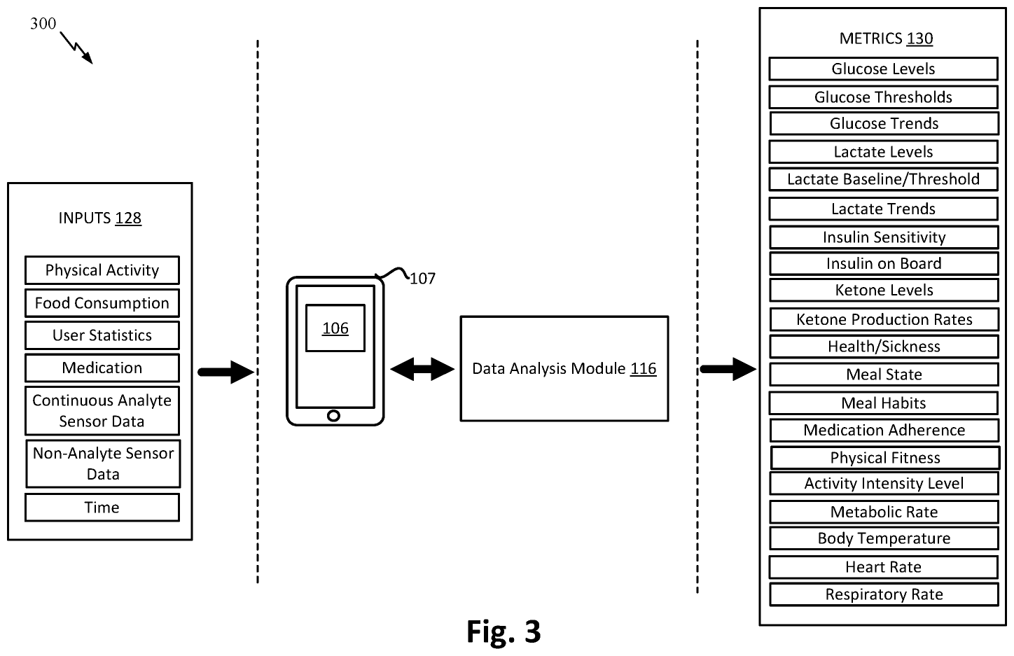

User profile 118 may include information collected about the user from application 106. For example, application 106 provides a set of inputs 128, including the analyte measurements associated with one or more analytes received from continuous analyte monitoring system 104 that are stored in user profile 118. In certain embodiments, inputs 128 provided by application 106 include other data in addition to analyte measurements. For example, application 106 may obtain additional inputs 128 through manual user input, one or more other non-analyte sensors or devices, other applications executing on display device 107, etc. Non-analyte sensors and devices include one or more of, but are not limited to, an insulin pump, respiratory sensor, sensors or devices provided by display device 107 (e.g., accelerometer, camera, global positioning system (GPS), heart rate monitor, etc.) or other user accessories (e.g., a smart watch or fitness tracker), or any other sensors or devices that provide relevant information about the user (e.g., sensors on exercise equipment). Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the physiological state of a user, trends associated with the health or state of a user, etc. In certain embodiments, metrics 130 may then be used by decision support engine 112 as input for providing guidance to a user. As shown, metrics 130 are also stored in user profile 118.

User profile 118 also includes demographic info 120, disease info 122, and/or medication info 124. In certain embodiments, such information may be provided through user input or obtained from certain data stores (e.g., electronic medical records, etc.). In certain embodiments, demographic info 120 may include one or more of the user's age, BMI (body mass index), ethnicity, gender, etc. In certain embodiments, disease info 122 may include information about one or more diseases of a user, including relevant information pertaining to the user's condition of diabetes and/or other conditions (e.g., liver disease, kidney disease, etc.). In certain embodiments, disease info 122 may also include the length of time since diagnosis, the level of disease control, level of compliance with disease management therapy, other types of diagnoses (e.g., heart disease, obesity), etc. In certain embodiments, disease info 122 may include other measures of health (e.g., heart rate, stress, sleep, etc.) or fitness (e.g., cardiovascular endurance, muscular strength and/or power, muscular endurance, and other measures of fitness), and/or the like.

In certain embodiments, medication info 124 may include information about the amount and type of a medication taken by a user. In certain embodiments, medication information may include information about the consumption of one or more drugs for management of the user's condition of diabetes, such as insulin (e.g., short-acting insulin, rapid-acting insulin (insulin aspart, insulin gluilisine, insulin lispro), intermediate-acting insulin (insulin isophane), long-acting insulin degludec, indulin detemir, insulin glargine, insulin), combination insulins), amylinomimetic drugs, alpha-glucosidase inhibitors (e.g., acarbose, miglitol), biguanides (e.g., metformin-alogliptin, metformin-canagliflozin, metformin-dapagliflozin, metformin-empagliflozin, metformin-glipizide, metformin-glyburide, metformin-linagliptin, metformin-pioglitazone, metformin-repaglinide, metformin-rosiglitazone, metformin-saxagliptin, metformin-sitagliptin), dopamine agonists (e.g., bromocriptine), dipeptidyl peptidase-4 (DPP-4) inhibitors (e.g., alogliptin, alogliptin-pioglitazone, linagliptin, linagliptin-empagliflozin, saxagliptin, sitagliptin, simvastatin), glucagon-like peptide-1 receptor agonists (GLP-1 receptor agonists) (e.g., albiglutide, dulaglutide, exenatide, liraglutide, semaglutide), meglitinides (e.g., nateglinide, repaglinide), sodium-glucose transporter (SGLT) 2 inhibitors (e.g., dapagliflozin, canagliflozin, empagliflozin, ertugliflozin), sulfonylureas (e.g., glimepiride, glimepiride-pioglitazone, glimepiride-rosiglitazone, gliclazide, glipizide, glyburide, chlorpropamide, tolazamide, tolbutamide), thiazolidinediones (e.g., rosiglitazone, pioglitazone), and other drugs. In certain embodiments, medication information may include information about the consumption of one or more drugs for management or treatment of other diseases or conditions of the user, including drugs for cholesterol, high blood pressure, heart disease, etc., in addition to supplements to promote general health and metabolism, such as vitamins.

Data stored in user profile 118 may maintain time series data collected for the user (e.g., the patient) over a period of time that the user utilizes continuous analyte monitoring system 104 and application 106. For example, analyte data for a user who has used continuous analyte monitoring system 104 and application 106 for a period of five years to manage the diabetic condition of the user may have time series analyte data associated with the user maintained in user profile 118 over the five-year period.

Further, data stored in user profile 118 may provide time series data collected over the lifetime of the user. For example, the data may include information associated with the user that indicates physiological states of the user, physical fitness level of the user, glucose levels of the user, lactate levels of the user, ketone levels of user, states/conditions of one or more organs of the user, habits of the user (e.g., alcohol consumption, activity levels, food consumption, etc.), medication(s) prescribed, etc., throughout the lifetime of the user.

In certain embodiments, user profile 118 is dynamic because at least part of the information that is stored in user profile 118 may be revised or updated over time and/or new information may be added to user profile 118 by decision support engine 114 and/or application 106. Accordingly, information in user profile 118 stored in user database 110 provides an up-to-date repository of information related to the user.

User database 110, in some embodiments, refers to a storage server that operates, for example, in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users, including users who similarly interact or have interacted in the past with application 106 on their own devices. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically data analysis module (DAM) 116 of decision support engine 114, can fetch inputs 128 from a user's profile 118 stored in user database 110 and compute one or more metrics 130 which can then be stored as application data 126 in the user's profile 118.

In certain embodiments, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored using timestamps. The timestamp associated with any piece of information stored in historical records database 112 may identify, for example, when the piece of information was obtained and/or updated.

Further, in certain embodiments, historical records database 112 may include data for one or more patients who are not users of continuous analyte monitoring system 104 and/or application 106. For example, historical records database 112 may include information (e.g., user profile(s)) related to one or more patients analyzed by, for example, a healthcare physician (or other known method), who may or may not be diagnosed with diabetes. Data stored in historical records database 112 may be referred to herein as population data.

Although depicted as separate databases for conceptual clarity, in some embodiments, user database 110 and historical records database 112 may operate as a single database. The single database may be a storage server that operates in a public or private cloud.

As previously mentioned, decision support system 100 is configured to provide a prediction of a current or future glycemic event for a user with diabetes who is engaging in physical activity, as well as specific treatment decisions or recommendations for diabetes management, using continuous analyte monitoring system 104 configured to continuously measure at least glucose and lactate levels. In certain embodiments, decision support engine 114 is configured to provide real-time or retrospective diabetes decision support, thus enabling an early prediction and/or provision of an early treatment recommendation for preventing or abating a predicted glycemic event. In particular, decision support engine 114 may be used to collect information associated with a user in user profile 118, to perform analytics thereon for detecting when the user is engaging in physical activity, determining the parameters of the physical activity, predicting a current or future glycemic event resulting from the physical activity, and providing one or more recommendations for treatment based, at least in part, on the prediction. User profile 118 may be accessible to decision support engine 114 over one or more networks (not shown) for performing such analytics.

In certain embodiments, decision support engine 114 may utilize one or more trained machine learning models capable of performing analytics on information that decision support engine 114 has collected/received from user profile 118. In the illustrated embodiment of FIG. 1, decision support engine 114 may utilize trained machine learning model(s) provided by a training server system 140. Although depicted as a separate server for conceptual clarity, in some embodiments, training server system 140 and decision support engine 114 may operate as a single server or system. That is, the model may be trained and used by a single server, or may be trained by one or more servers and deployed for use on one or more other servers or systems.

Training server system 140 is configured to train the machine learning model(s) using training data, which may include data (e.g., from user profiles) associated with one or more diabetic patients (e.g., users or non-users of continuous analyte monitoring system 104 and/or application 106). The training data may be stored in historical records database 112 and may be accessible to training server system 140 over one or more networks (not shown) for training the machine learning model(s).

The training data refers to a dataset that has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model. As an illustrative example, each relevant characteristic of a user, which is reflected in a corresponding data record, may be a feature used in training the machine learning model. Such features may include age, gender, fitness, normal glucose ranges, normal lactate ranges (e.g., lactate baseline and/or peak), start and end times associated with physical activity, duration of physical activity, physical activity type and/or intensity (e.g., based on a delta in glucose levels and/or a delta in lactate levels), time of and/or duration associated with a glycemic event, glucose information associated with a glycemic event (e.g., glucose levels associated with a hypo- or hyperglycemic events, etc.), treatments taken, glucose information subsequent to treatment, etc.

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, the model(s) may be iteratively refined to generate accurate predictions of a glycemic event for a patient engaging in physical activity.

As illustrated in FIG. 1, training server system 140 deploys these trained model(s) to decision support engine 114 for use during runtime. For example, decision support engine 114 may obtain user profile 118 associated with a user and stored in user database 110, use information in user profile 118 as input into the trained model(s), and output a prediction indicative of the current or future physical activity-induced glycemic event for the user (e.g., shown as output 144 in FIG. 1). Output 144 generated by decision support engine 114 may also provide one or more recommendations for treatment based on the prediction. Output 144 may be provided to the user (e.g., through application 106), to a caretaker of the user (e.g., a parent, a relative, a guardian, a teacher, a physical therapist, a fitness trainer, a nurse, etc.), to a physician or healthcare provider of the user, or any other individual that has an interest in the wellbeing of the user for purposes of improving the health of the user, such as, in some cases by effectuating recommended treatment. Output 144 generated by decision support engine 114, in addition to the actual glycemic event and/or treatment taken by a user, is stored in user database 110 and is utilized to train or re-train the trained model(s). In certain embodiments, output 144 generated by decision support engine 114, which may be indicative of the physiological state of a user and/or current treatment recommended to a user, may be stored in user profile 118.

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise one or more sensors for detecting and/or measuring analytes. The continuous analyte sensor 202 may be a multi-analyte sensor configured to continuously measure two or more analytes, such as at least glucose and lactose, or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes in the user. The data stream may include raw data signals, which are then converted into a calibrated and/or filtered data stream used to provide estimated analyte value(s) to the user.

In certain embodiments, the continuous analyte sensor 202 may be a multi-analyte sensor, configured to continuously measure multiple analytes in a user's body. For example, in certain embodiments, the continuous multi-analyte sensor 202 may be a single sensor configured to measure glucose, lactate, ketones, glycerol, potassium (e.g., hyperkalemia), sodium, and/or CO₂ or anion-gap in the user's body.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enable measurement of levels of analyte(s) via continuous analyte sensor(s) 202. For example, sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 may include a display such as a touchscreen display 212, 222, 232, and/or 242 for displaying sensor data to a user and/or for receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or for receiving user inputs. Display devices 210, 220, 230, and 240 may be examples of display device 107 illustrated in FIG. 1 used to display sensor data to a user of the system of FIG. 1 and/or to receive input from the user.

In some embodiments, one, some, or all of the display devices are configured to display or otherwise communicate the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor information.

As mentioned, sensor electronics module 204 may be in communication with a medical device 208. Medical device 208 may be a passive device in some example embodiments of the disclosure. For example medical device 208 may be an insulin pump for administering insulin to a user. For a variety of reasons, it may be desirable for such an insulin pump to receive and track glucose values transmitted from continuous analyte monitoring systems 104, where continuous analyte sensor 202 comprises at least a glucose sensor and a lactate sensor.

Further, as mentioned, sensor electronics module 204 may also be in communication with other non-analyte sensors 206. Non-analyte sensors 206 may include, but are not limited to, an altimeter sensor, an accelerometer sensor, a global positioning system (GPS) sensor, a temperature sensor, a respiratory sensor, electromyogram (EMG) sensor, a galvanic skin response (GSR) sensor, an impedance sensor, etc. Non-analyte sensors 206 may also include monitors such as heart rate monitors, blood pressure monitors, pulse oximeters, caloric intake monitors, and medicament delivery devices. One or more of these non-analyte sensors 206 may provide data to decision support engine 114 described further below. In some aspects, a user may manually provide some of the data for processing by training server system 140 and/or decision support engine 114 of FIG. 1.

In certain embodiments, non-analyte sensors 206 may further include sensors for measuring skin temperature, core temperature, sweat rate, and/or sweat composition.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous glucose sensor 202 to form a glucose/temperature sensor used to transmit sensor data to the sensor electronics module 204 using common communication circuitry. As another illustrative example, a non-analyte sensor, e.g., a temperature sensor, may be combined with a multi-analyte sensor 202 configured to measure glucose and lactate to form a glucose/lactate/temperature sensor used to transmit sensor data to the sensor electronics module 204 using common communication circuitry.

One or more of continuous analyte monitoring system 104, the plurality of display devices, medical device(s) 208, and/or non-analyte sensor(s) 206 may be configured to communicate together wirelessly using one of a variety of wireless communication technologies (e.g., Wi-Fi, Bluetooth, Near Field Communication (NFC), cellular, etc.). In certain embodiments, a wireless access point (WAP) may be used to couple one or more of continuous analyte monitoring system 104, the plurality of display devices, medical device(s) 208, and/or non-analyte sensor(s) 206 to one another. For example, the WAP may provide Wi-Fi, Bluetooth, and/or cellular connectivity among these devices. NFC may also be used among devices depicted in diagram 200 of FIG. 2.

FIG. 3 illustrates example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors, other applications executing on display device 107, an EMR system, etc.). As mentioned previously, in certain embodiments, inputs 128 may be processed by DAM 116 to output a plurality of metrics, such as metrics 130. Inputs 128 and metrics 130 may be used by training server system 140 and decision support engine 114 to both train and deploy one or more machine learning models for predicting glycemic events of users engaging in physical activity.

In certain embodiments, starting with inputs 128, physical activity information may include any information surrounding activities, such as activities requiring physical exertion by the user. For example, physical activity information may include information for physical activities ranging from a relatively low intensity of physical exertion (e.g., walking, standing, passive stretching, etc.) to a relatively high intensity of physical exertion (e.g., sprinting, weight lifting, action sports), including aerobic and anaerobic exercises. Such information may be based on continuous analyte sensor data measured by continuous analyte sensor(s) 202, non-analyte sensor data measured by non-analyte sensor(s) 206 (e.g., non-analyte sensor data input from an accelerometer, a heart rate monitor, a respiration rate sensor, etc.), user statistics stored in user profile 118, etc. For example, in certain embodiments, physical activity information may be based on glucose data, lactate data, accelerometer data, step rate data, exercise equipment power meter data, GPS data, heart rate data (e.g., heart rate reserve and heart rate variability (HRV)), electrocardiogram (EKG) data, EMG data, respiration rate data, temperature data, blood pressure data, galvanic skin response data, oxygen uptake data, sleep data, impedance data, etc., associated with the user, and may be provided by continuous analyte sensors or non-analyte sensors, as described above. In certain embodiments, physical activity information may be provided, for example, by an accelerometer sensor on a wearable device such as a watch, fitness tracker, and/or patch. In certain embodiments, physical activity information may also be provided through manual user input.

In certain embodiments, food consumption is also provided as an input. Food consumption information may include information about one or more of meals, snacks, and/or beverages, such as one or more of the size, content (carbohydrate, fat, protein, etc.), sequence of consumption, and time of consumption. In certain embodiments, food consumption may be provided by a user through manual entry, by providing a photograph through an application that is configured to recognize food types and quantities, and/or by scanning a bar code or menu. In various examples, meal size may be manually entered as one or more of calories, quantity (“three cookies”), menu items (“Royale with Cheese”), and/or food exchanges (1 fruit, 1 dairy). In some examples, meal information may be received via a convenient user interface provided by application 106. In further examples, meal size may be passively determined using continuous analyte sensor data. For example, lactate levels may indicate the size of a meal (e.g., larger meals produce larger spikes in lactate levels). Such lactate response data may be distinct from elevated lactate levels caused by physical activity, since lactate spikes caused by food consumption precede changes in glucose (e.g., glucose spikes), and may be slow to return to baseline, unlike exercise.

In certain embodiments, user statistics, such as one or more of age, height, weight, BMI, body composition (e.g., % body fat), stature, build, or other information may also be provided as an input. Other examples of user statistics may include historical exercise data, such as race (or other exercise event) results, normal training paces, and historical biomarker response to physical activity (e.g., heart rate or typical average glucose for running at a given pace or riding at a given power level). In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment/medication information is also provided as an input. Medication information may include information about the type, dosage, and/or timing of when one or more medications have been or are to be taken by the user. Treatment information may include information regarding different lifestyle habits recommended by the user's physician. For example, the user's physician may recommend a user drink alcohol sparingly, exercise for a minimum of thirty minutes a day, or cut calories by 500 to 1,00 calories daily to improve general health. In certain embodiments, treatment/medication information may be provided through manual user input.

In certain embodiments, analyte sensor data may also be provided as input, for example, through continuous analyte monitoring system 104. In certain embodiments, analyte sensor data may include glucose data (e.g., a user's time-stamped glucose values, trends, patterns, etc.) measured by at least a glucose sensor or a multi-analyte sensor in continuous analyte monitoring system 104. In certain embodiments, analyte sensor data may include lactate data (e.g., a user's time-stamped lactate values, trends, patterns, etc.) measured by at least a lactate sensor or a multi-analyte sensor in continuous analyte monitoring system 104. In certain embodiments, analyte sensor data may include ketone data (e.g., a user's time-stamped ketone values, trends, patterns, etc.) measured by at least a ketone sensor or a multi-analyte sensor in continuous analyte monitoring system 104. In certain embodiments, analyte sensor data may include data extracted from intermittent blood samples by continuous analyte monitoring system 104.

In certain embodiments, input may also be received from non-analyte sensors, such as non-analyte sensors 206 described with respect to FIG. 2. Input from such non-analyte sensors 206 may include information related to a heart rate, a respiration rate, oxygen saturation, or a body temperature (e.g. to detect illness, physical activity, etc.) of a user as well as trends and/or patterns thereof. In certain embodiments, electromagnetic sensors may also detect low-power radio frequency (RF) fields emitted from objects or tools touching or near the object, which may provide information about user activity or location.

In certain embodiments, input received from non-analyte sensors may include input relating to a user's insulin delivery. In particular, input related to the user's insulin delivery may be received, via a wireless connection on a smart pen, via user input, and/or from an insulin pump. Insulin delivery information may include one or more of insulin volume, time of delivery, etc. Other parameters, such as insulin action time or duration of insulin action, may also be received as inputs.

In certain embodiments, time may also be provided as an input, such as time of day or time from a real-time clock. In certain embodiments, one or more of inputs 128 as well as one or more of metrics 130 may be timestamped. For example, in certain embodiments, input analyte data may be timestamped to indicate a date and time when the analyte measurement was taken for the user.

User input of any of the above-mentioned inputs 128 may be provided through a user interface, such a user interface of display device 107 of FIG. 1. As described above, in certain embodiments, DAM 113 determines or computes the user's metrics 130 based on inputs 128. An example list of metrics 130 is shown in FIG. 3.

In certain embodiments, glucose levels may be determined from sensor data (e.g., blood glucose measurements obtained from a continuous glucose sensor of continuous analyte monitoring system 104). For example, glucose levels refer to time-stamped glucose measurements or values that are continuously generated and stored over time. In some examples, glucose levels may also be determined, for example, based upon historical data in particular situations, e.g., given a combination of food consumption, insulin, and/or physical activity.

In certain embodiments, glucose trends may be determined based on glucose levels over certain periods of time.

In certain embodiments, a normal glucose range may be determined from sensor data (e.g., blood glucose measurements obtained from continuous analyte monitoring system 104). A normal glucose range may be indicative of a range within which the user does not experience any symptoms (e.g., hyper- or hypoglycemic-related symptoms) under controlled conditions, such as when the user is fasting, during a given time period post meals, etc. The range may have a minimum and/or a maximum. For example, in certain embodiments, the range may have a minimum normal glucose concentration value. In certain embodiments, the range may have a maximum normal glucose concentration value. A user's normal glucose range may be expected to remain constant or change gradually over time. Further, each user may have a different normal glucose range. In certain embodiments, a normal glucose range may be determined by determining a minimum and/or maximum average value of glucose over a specified amount of time where fluctuations are not expected. In certain embodiments, a normal glucose range for a user may be determined over a period of time when the user is sleeping, sitting in a chair, or other periods of time where the user is sedentary. In certain embodiments, DAM 116 may continuously or periodically calculate a normal glucose range and time-stamp and store the corresponding information in the user's profile 118.

In still other embodiments, a normal glucose range may be determined from population data (e.g., from data records or samples of historical patients with diabetes). In such embodiments, each user may have personalized, i.e., customized, acceptable minimum and/or maximum glucose values, which may be determined by methods similar to those described above (e.g., determining a range within which the user does not experience any symptoms under controlled conditions, such as when the user is fasting, during a given time period post meals).

In certain embodiments, lactate levels may be determined from sensor data (e.g., lactate measurements obtained from continuous analyte monitoring system 104). For example, lactate levels refer to time-stamped lactate measurements or values that are continuously generated and stored over time. In some examples, lactate levels may also be determined, for example, based upon historical data in particular situations, e.g., given a combination of food consumption and/or physical activity.

In certain embodiments, lactate trends may be determined based on lactate levels over certain periods of time. In certain embodiments, lactate trends may be determined based on lactate production rates and/or calculated lactate clearance rates over certain periods of time.

In certain embodiments, a lactate baseline may be determined from sensor data (e.g., lactate measurements obtained from continuous analyte monitoring system 104). A lactate baseline may be indicative of the user's lactate values during periods where fluctuations in lactate production/clearance are typically not expected. A user's lactate baseline is typically expected to remain constant or change gradually over time. Further, each user may have a different lactate baseline. In certain embodiments, a lactate baseline may be determined by determining an average of lactate measurements over a specified amount of time where fluctuations are not expected. In certain embodiments, a lactate baseline for a user may be determined over a period of time when the user is sleeping, sitting in a chair, or other periods of time where the user is sedentary. In certain embodiments, DAM 116 may continuously or periodically calculate a lactate baseline and time-stamp and store the corresponding information in the user's profile 118.

In certain embodiments, a “lactate threshold” may be determined from sensor data (e.g., lactate measurements obtained from continuous analyte monitoring system 104). A lactate threshold may be indicative of the lactate value of a user at which lactate production exceeds lactate clearance. This may be caused by the user engaging in high intensity, anaerobic activity. Each user may have a different lactate threshold. In certain embodiments, a lactate threshold may be determined by determining a lowest lactate value during a specified amount of time where lactate levels increase exponentially (i.e., rapidly). In certain embodiments, lactate threshold may be determined by determining a highest lactate value before an increasing work rate of the user leads to exponentially increasing lactate levels. In certain embodiments, a lactate threshold for a user may be determined over a period of time when the user is engaging in physical activity, such as moderate to high intensity physical activity. In certain embodiments, DAM 116 may continuously or periodically calculate a lactate threshold and time-stamp and store the corresponding information in the user's profile 118.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin, and such information may provide important actionable insights for users. For example, improving insulin sensitivity for a user may help to reduce insulin resistance in the user. In certain embodiments, insulin sensitivity may be utilized to adjust a user's recommended dose of insulin.

In certain embodiments, insulin sensitivity may be determined based on an active insulin sensitivity assessment. For example, the user may be requested, e.g., by decision support system 100, to consume a known amount of a substance that is expected to cause a spike in glucose, and a known amount of insulin, after which glucose levels and/or trends of the user may be monitored for a predetermined time period of time to determine insulin sensitivity of the patient. Such an active insulin sensitivity assessment may be performed at baseline (e.g., at rest), before engagement in physical activity, or post-physical activity, with a limited dose of insulin. In some examples, insulin sensitivity may be additionally determined or adjusted based on other considerations, such as adjustments to other analytes, or other substances that cause an insulin response or may be expected to effect the insulin clearance rate.

In certain embodiments, insulin sensitivity may be calculated using inputs over a long period of time, and may reflect a long term average health state. In other embodiments, insulin sensitivity may be estimated frequently using real-time data, historical data, or a combination thereof, and changes in insulin sensitivity may be used to estimate changes in metabolic health. In certain embodiments, insulin resistance may be estimated by monitoring analyte data in real time (e.g., insulin, glucose, lactate, glycerol, ketones, potassium, etc.), particularly in response to known exercise or food challenges.

In certain cases, a user may experience hyperlactatemia as a result of physical activity, and lactate levels may be slow to return to baseline. In such cases, the user may experience hyperglycemia and acute insulin resistance due to the hyperlactatemia. Upon normalization of the user's lactate levels, the patient's insulin sensitivity may return to the user's prior level of insulin sensitivity. Accordingly, monitoring lactate levels and/or trends may inform insulin sensitivity measurements. In addition to being responsive to physical activity and short term dietary changes, insulin sensitivity also appears to precede metabolic syndrome, type 2 diabetes, and heart disease.

In certain embodiments, insulin on board may be determined using non-analyte sensor data input (e.g., insulin delivery information) and/or known or learned (e.g. from user data) insulin time action profiles, which may account for both basal metabolic rate (e.g., update of insulin to maintain operation of the body) and insulin usage driven by activity or food consumption.

In certain embodiments, ketone levels may be determined from sensor data (e.g., ketone measurements obtained from continuous analyte monitoring system 104). In certain embodiments, ketone levels may be determined from surrogate sensor data, e.g., changes in levels or trends of lactate and/or glucose, and/or other non-analyte sensor data, such as changes in heart rate, blood pressure, and/or other non-analyte metrics. In certain embodiments, ketone levels may be expressed as a metric of whether or not the user is in ketosis. In particular, ketosis is a metabolic state in which there's a high concentration of ketones in the user's blood. Ketosis can be an indicator of low glycogen stores. In patients with diabetes, elevated blood ketones indicate a relative or absolute insulin deficiency.

In certain embodiments, a ketone production rate may be determined from sensor data (e.g., ketone measurements obtained from continuous analyte monitoring system 104). In particular, ketones (chemically known as ketone bodies) are byproducts of the breakdown of fatty acids. Glucose (e.g., blood sugar) is the preferred fuel source for many cells in the body; however, when there is limited access to glucose by the cells, fat may be broken down for fuel, thereby producing ketones as byproducts. In certain embodiments, a ketone production rate may be determined by assessing the increase in ketone levels over a specified amount of time.

In certain embodiments, health and sickness metrics may be determined, for example, based on one or more of user input (e.g., pregnancy information or known sickness information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Better/healthier meals may be defined as those that do not drive glucose levels of a user out of a normal glucose range for the user (e.g., 70-180 mg/dL or the user's desired range). Also, the more the user's food consumption adheres to a certain time schedule, the closer their meal habit metric will be to 1, in the example. In certain embodiments, the meal habit metrics may indicate whether a user has been consistently participating in a ketogenic diet (e.g., a low-carb, moderate protein, higher-fat diet) based on meals, snacks, or beverages consumed by the user over a certain period of time. In another example, the meal habit metrics may reflect the contents of a patient's meals where, e.g., three numbers may indicate the percentages of carbohydrates, proteins and fats.

In certain embodiments, medication adherence is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage).

In certain embodiments, physical fitness metrics may indicate the user's level of physical fitness. In certain embodiments, the physical fitness metric may be determined, for example, based on one or more inputs 128, such as one or more of physical activity information, food consumption, user statistics (such as height and weight), continuous analyte sensor data (e.g., lactate threshold), non-analyte sensor data, etc. In certain embodiments, the physical fitness metric is determined based on input from activity sensors or other physiologic sensors, as well as type, intensity, and/or frequency of physical activities.

In certain embodiments, activity intensity level metrics may indicate the intensity level with which the user is performing the activity. For example, activity intensity level metrics may include information indicating that the user is engaging in low intensity physical activity, low-to-moderate intensity physical activity, moderate intensity physical activity, moderate-to-high intensity physical activity, and/or high intensity physical activity, which may all impact the user's glucose levels. In certain embodiments, activity intensity level metrics may be calculated by DAM 116 based on one or more of inputs 128, such as one or more of physical activity information, non-analyte sensor data, time, user statistics, etc. For example, in certain embodiments, activity intensity level metrics may be determined based on physical activity information, such as input from an activity sensor on a fitness tracker or other physiologic sensors. In certain embodiments, activity intensity level metrics may be determined based on input from other non-analyte sensors, such as an accelerometer, exercise equipment sensor (e.g., a power meter), GPS device, heart rate monitor, EKG device, EMG device, respiration monitor, temperature monitor, blood pressure monitor, pulse oximeter, etc. In certain embodiments, activity intensity level metrics may be determined based on skin temperature, core temperature, sweat rate, and/or sweat composition. In certain embodiments, activity intensity level metrics may be determined based on user statistics, such as information stored in user profile 118 or provided through manual user input. In further embodiments, activity level metrics may be based on continuous analyte sensor data measured by continuous analyte sensor(s) 202, such as lactate (e.g., lactate threshold, lactate production/clearance rates, etc.) and/or glucose levels.

In certain embodiments, metabolic rate is a metric that may indicate or include a basal metabolic rate (e.g., energy consumed at rest) and/or an active metabolism (e.g., energy consumed by physical activity, such as physical exertion). In some examples, basal metabolic rate and active metabolism may be tracked as separate outcome metrics. In certain embodiments, the metabolic rate may be calculated by DAM 116 based on one or more of inputs 128, such as one or more of physical activity information, continuous analyte sensor data, non-analyte sensor data, time, etc.

In certain embodiments, body temperature metrics may be calculated by DAM 116 based on inputs 128, and more specifically, non-analyte sensor data from a temperature sensor. In certain embodiments, heart rate metrics may be calculated by DAM 116 based on inputs 128, and more specifically, non-analyte sensor data from a heart rate sensor. In certain embodiments, respiratory metrics may be calculated by DAM 116 based on inputs 128, and more specifically, non-analyte sensor data from a respiratory rate sensor.

### Example Methods and Systems for Predicting a Glycemic Event of a Patient Engaging in Physical Activity Using Continuously Monitored Analyte Data

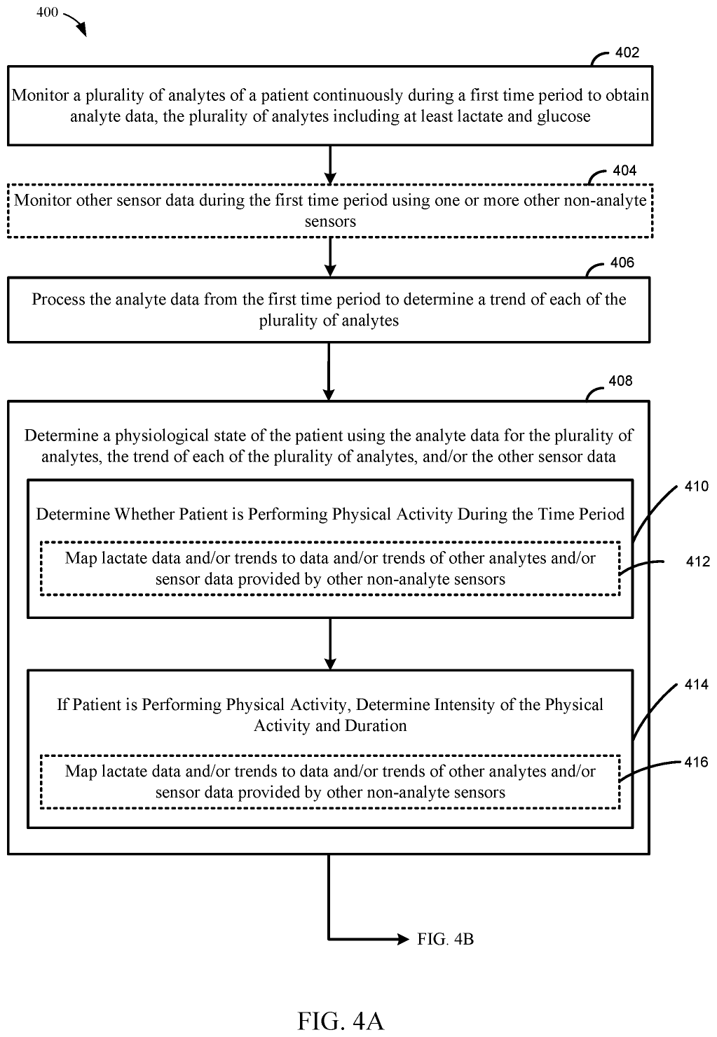

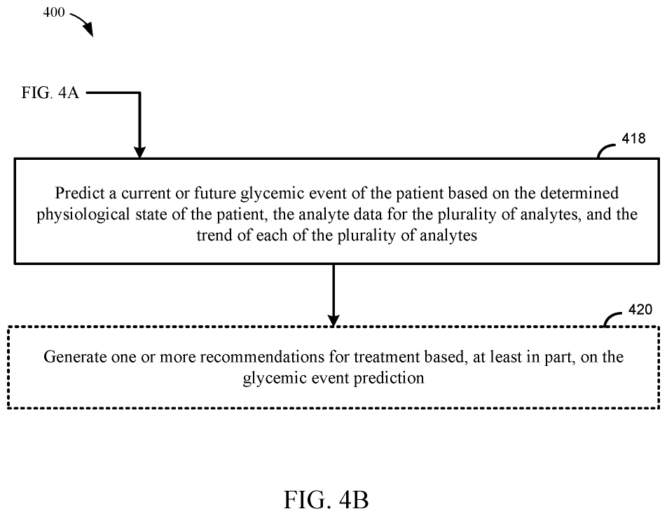

FIGS. 4A and 4B illustrate a flow diagram of an example method 400 for providing decision support using one or more continuous analyte sensors configured to measure or otherwise indicate, at least, a user's glucose and lactate levels, in accordance with certain aspects of the present disclosure. For example, method 400 may be performed to provide decision support to a user using a continuous analyte monitoring system 104, as illustrated in FIGS. 1 and 2. Method 400 may be performed by decision support system 100 to collect data, including for example, analyte data, patient information, and non-analyte sensor data mentioned above, to (1) detect (e.g., automatically) the patient engaging in physical activity; (2) determine relevant parameters of the physical activity engaged by the patient (e.g., intensity, duration, and/or type of physical activity); (3) predict a current or future physical activity-induced glycemic event based, in part, on the relevant parameters associated with the physical activity engaged by the patient, and (4) make patient-specific treatment decisions or recommendations for diabetes. In other words, the decision support system presented herein may offer information to direct and help improve care for patients with diabetes when and/or after engaging in physical activity. Method 400 is described below with reference to FIGS. 1 and 2 and their components.

At block 402, method 400 begins by continuously monitoring a plurality of analytes of a patient, such as user 102 illustrated in FIG. 1, during a time period to obtain analyte data, the plurality of analytes including at least glucose and lactate. Block 402 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a continuous glucose sensor 202 to measure the user's glucose levels and a continuous lactate sensor 202 to measure the user's lactate levels. Alternatively, continuous analyte monitoring system 104 may comprise a continuous multi-analyte sensor to measure both the user's lactate as well as glucose levels. As previously discussed, in certain situations (e.g., periods of anaerobic cellular respiration and high metabolism during moderate-high intensity physical activity), the presence of lactate may better indicate the type and intensity of physical activity engaged by a user than other analytes, such as for example, glucose levels alone. Thus, continuous monitoring of lactate, in addition to glucose, may be needed to assess the physical activity engaged by a user in order to accurately predict glycemic events resulting from the user engaging in such physical activity.

In certain embodiments, continuous monitoring of lactate may be utilized to optimize performance of a continuous analyte monitoring system, such as continuous analyte monitoring system 104, by enabling more immediate predictions of hyperglycemic events via adjustments to glucose sampling rates. High rates of change in lactate levels may, at least in certain cases, indicate or predict high rates of change in glucose levels. For example, high rates of change in lactate levels may indicate performance of physical activity or a change in the intensity level of a physical activity, as described above, which may cause sudden or high rates of change in glucose levels, thus informing predictions of glycemic events. A high rate of change in lactate levels may be partially defined as a minimum, e.g., threshold, positive or negative delta in lactate levels. Accordingly, lactate levels may be utilized to optimize sampling of glucose during, e.g., period of physical activity.

In certain examples, upon continuous analyte monitoring system 104 determining an abrupt delta in lactate levels, continuous analyte monitoring system 104 may utilize, or adjust, a predicted interstitial lag time for interstitial analyte measurements, including glucose and lactate measurements. Generally, changes in interstitial analyte levels lag slightly behind, or are delayed, in relation to changes in blood analyte levels, due to the time required for analytes to diffuse from capillaries to surrounding tissue. This interstitial lag time may change in length as volume of distribution is shifted when a user engages in physical activity. Thus, detection of an abrupt delta in lactate levels, which may indicate engagement of the user in physical activity and, therefore, a shift in volume of distribution, may further indicate a change in interstitial lag time for analyte measurements. Accordingly, upon determining such an abrupt change in lactate levels, continuous analyte monitoring system 104 may adjust a previously predicted interstitial lag time, or may utilize/determine a new predicted interstitial lag time, which is then factored into the continuous measurements and/or predictions of the user's analyte levels. In certain embodiments, a predicted interstitial lag time may be continuously factored into continuous analyte measurements of the user. In certain embodiments, a predicted interstitial lag time may be factored into continuous analyte measurements of the user only when an abrupt delta in lactate levels is determined, and/or when elevated lactate levels are determined.

In certain embodiments, the abrupt delta in lactate levels utilized to adjust the predicted interstitial lag time is based on a real-time slope of the user's lactate levels and/or a rate-of-change of the user's lactate levels. In such embodiments, upon the real-time slope and/or the rate-of-change meeting or exceeding a predetermined threshold, continuous analyte monitoring system 104 may then adjust the predicted interstitial lag time for interstitial analyte measurements.

In certain examples, upon continuous analyte monitoring system 104 determining an abrupt delta in lactate levels (thus indicating potential impending abrupt changes in glucose levels), continuous analyte monitoring system 104 may further increase the sampling rate of continuous glucose sensor 202 in order to facilitate more accurate glucose measurements. By increasing the rate of sampling, the number of data points for analysis is increased, thereby facilitating measurements that are more accurately indicative of the patient's real-time glucose levels. For example, if a baseline sampling rate is 1 sample per minute, then upon continuous analyte monitoring system 104 detecting a high rate of change in lactate levels, the sampling rate of the continuous glucose sensor 202 may be increased to 1 sample per 30 seconds, or 1 sample per 20 seconds, or 1 sample per 10 seconds, or 1 sample per 5 seconds, or 1 sample per 3 seconds, etc.

In certain examples, upon continuous analyte monitoring system 104 determining an abrupt delta in lactate levels (thus indicating potential abrupt changes in glucose levels), continuous analyte monitoring system 104 may further increase the data transmission rate from continuous glucose sensor 202 to a display device, e.g., display device 107, to provide more advanced warning to a user of a current or future glycemic event. Sending sensor readings to the display device 107 more frequently facilitates earlier analysis of measurements, and thus, earlier prediction of glycemic events, thus enabling the earlier provision of a warning and/or recommendation to the user. For example, if a baseline data transmission rate is 1 transmission per 5 minutes, then upon continuous analyte monitoring system 104 detecting a high rate of change in lactate levels, the data transmission rate of the continuous glucose sensor 202 may be increased to 1 transmission per 3 minutes, or 1 transmission per 1 minute, or 1 transmission per 30 seconds, etc.

While the main analytes for measurement described herein are glucose and lactate, in certain embodiments, other analytes may be considered. In particular combining glucose and lactate measurements with additional analyte data may help to further inform the analysis around predicting glycemic events resulting from physical activity. For example, monitoring additional types of analytes, such as ketones measured by continuous analyte monitoring system 104, may provide additional insight into the type or intensity of a physical activity and/or supplement information used to determine optimal treatment for preventing or abating a glycemic event induced by physical activity.

The additional insight gained from using a combination of analytes, and not just glucose and lactate, may increase the accuracy of glycemic event prediction. For example, the probability of accurately predicting a glycemic event may be a function of a number of analytes measured for a user. More specifically, in certain examples, a probability of accurately predicting a glycemic event using only glucose and lactate (in addition to other non-analyte data) may be less than a probability of accurately predicting a glycemic event using glucose, lactate, and another analyte (in addition to other non-analyte data).

In certain embodiments described herein, analyte combinations, e.g., measured and collected by one (e.g., multianalyte) or more sensors, for glycemic event prediction and diabetes decision support, include glucose, lactate, and ketones; however, other analyte combinations may be considered. For example, in certain embodiments, at block 402, continuous analyte monitoring system 104 may continuously monitor glucose levels, lactate levels, and ketone levels of a user during a time period. In such embodiments, the measured ketone concentrations may be used to further inform analysis for predicting a glycemic event, such as hyperglycemia and/or diabetic ketoacidosis (DKA).

In particular, insulin mediates precise regulation of glucose metabolism and plasma concentrations by promoting glucose uptake by skeletal muscle, liver, and adipose tissue. Accordingly, when insulin is low, there is limited glucose uptake by the skeletal muscle, liver, and adipose tissue, and glucose levels rise. Such limited access to glucose also causes the breakdown of fat for fuel (e.g., ketogenesis), which produces a buildup of acids in the blood stream called ketones (e.g., ketone bodies). If untreated, the increased concentration of ketones in the blood leads to DKA. Thus, ketone levels, when combined with other analyte measurements and/or non-analyte measurements, may be indicative of physical activity-induced events, such as hyperglycemia and DKA. In certain embodiments, ketone levels and/or the occurrence of ketosis may be utilized to determine a level of glycogen stored in cells of the patient, and thus may inform meal recommendations provided for the patient. For example, a determination of a high ketone levels may indicate low glycogen levels of the patient, and thus, that the patient may need to consume glucose or a carbohydrate-heavy meal to normalize glycogen stores.

In certain embodiments, AI models, such as machine learning models, may be used to provide predictions of physical activity-induced glycemic events and real-time or retrospective diabetic decision support. In certain embodiments, such models may be configured to use input from one or more sensors measuring multiple analytes to provide predictions of physical activity-induced glycemic events. Accordingly, given the interaction of such comorbidities (e.g., as discussed with respect to the example for a user with DKA and/or hyperglycemia above), parameters and/or thresholds of such algorithms or models may be altered based, at least in part, on a number of analytes being measured for input to reflect the knowledge attained from each of the other analytes being measured or the morbidities associated with the additional analytes being measured.

In addition to continuously monitoring one or more analytes of a user during a time period to obtain analyte data at block 402, optionally, in certain embodiments, at block 404, method 400 may also include monitoring non-analyte data during the time period using one or more non-analyte sensors or devices. Block 404 may be performed by non-analyte sensors 206 and/or medical device 208 of FIG. 2, in certain embodiments.

As mentioned previously, non-analyte sensors and devices may include one or more of, but are not limited to, an insulin pump, a respiratory sensor, a heart rate monitor, electromyogram (EMG) sensor, an accelerometer, an altimeter sensor, a temperature sensor (e.g., thermometer), a blood pressure monitor, a galvanic skin response (GSR) sensor, a pulse oximeter, a caloric intake monitor, sensors or devices provided by display device 107 (e.g., accelerometer, camera, global positioning system (GPS), etc.), or any other sensors or devices that provide relevant information about the user and/or the physical activity being engaged by the user. In certain embodiments, non-analyte sensors and/or devices may further include sensor for measuring skin temperature, core temperature, sweat rate, and/or sweat composition. Such non-analyte sensors and/or devices may be worn or used by a user to aid in detection of periods of physical activity engaged by the user, in addition to utilizing analyte data for multiple analytes.

Metrics, such as metrics 130 illustrated in FIG. 3, may be calculated using measured data from each of these non-analyte sensors. As further illustrated in FIG. 3, metrics 130 calculated from non-analyte sensor or device data may include metabolic rate, body temperature, heart rate, respiratory rate, etc. Such metrics 130, though not shown, may further include heart rate reserve, heart rate variability (HRV), blood pressure, GSR, oxygen uptake (VO₂), sleep metrics, etc. In certain embodiments, metrics may further include skin temperature, core temperature, sweat rate, and/or sweat composition. In certain embodiments, described in more detail below, metrics 130 calculated from non-analyte sensor or device data may be used to further inform analysis for predicting physical activity-induced glycemic events and for providing real-time or retrospective diabetes decision support.

In certain embodiments, the non-analyte sensor data may be utilized as a surrogate for the user's lactate levels continuously monitored at block 402. For example, in certain embodiments, non-analyte sensor data, such as heart rate data (e.g., current or historical), may be correlated with the user's continuously monitored lactate data (e.g., current or historical). Thereafter, the non-analyte sensor data may be utilized in place of continuous lactate measurements, thereby eliminating the need for a continuous lactate sensor and/or a continuous multi-analyte sensor configured to measure lactate.

In certain embodiments, the non-analyte sensor data may be utilized to confirm or modify determinations based on analyte data and related to physical activity. For example, a slope of lactate levels and/or other analyte data may be monitored in combination with non-analyte data such as, e.g., GPS data and/or accelerometer data. In such an example, where a slope of lactate levels increases rapidly but the GPS data and/or accelerometer data suggests more moderate physical activity, then a prediction of physical activity may be adjusted to reflect more moderate physical activity, as the rapid slope of lactate may be caused by, e.g., a lack of a warm-up period. If, on the other hand, the non-analyte data suggests the user is engaging in a more intense physical activity than the lactate slope suggests, the prediction may be adjusted to reflect a more intense physical activity.

At block 406, method 400 continues by processing the analyte data from the time period to determine one or more trends of each of the plurality of analytes being continuously monitored. For example, as mentioned above, the analyte measurements taken during the time period, including at least glucose levels and lactate levels, are processed to determine directional trends, which may include data such as a minimum measurement of an analyte during the time period, a maximum measurement of an analyte during the time period, a direction of change (e.g., upward rise or downward fall) of analyte levels during the time period, rate of change of analyte levels during the time period, etc. For example, lactate trends determined at block 406 may include lactate production rates, lactate clearance rates, lactate threshold, etc., over a certain period of time. Block 406, in certain embodiments, may be performed by decision support engine 114.

At block 408, decision support engine 114 determines a physiological state of the patient, e.g., the user, during the time period, based on the analyte data for each of the plurality of analytes, the trend(s) of each of the plurality of analytes, and in certain embodiments, the non-analyte sensor or device data. The physiological state of the patient refers to the general condition or bodily state of the user, including any physical stresses thereon.

In certain embodiments, determining the physiological state of the user includes determining whether the user is engaging in physical activity during the time period, as shown at block 410 of FIG. 4A. The performance of physical activity may be determined utilizing the analyte data and/or trend(s) of each of the plurality of analytes alone, or may be determined using the analyte data and/or trend(s) of each of the plurality of analytes in combination with non-analyte sensor or device data. For example, lactate levels of a user typically increase when the user exercises or engages in other forms of physical activity, as a result of decreased availability of oxygen to the user's muscles for breaking down glucose for energy. Thus, in certain embodiments, decision support engine 114 may determine the performance of physical activity by detecting elevated or irregular lactate levels, such as a lactate spike, a change in lactate levels, and/or a rate of change in lactate levels exceeding predetermined thresholds.

To rule out elevated or irregular lactate levels and/or trends caused by other events (e.g., eating, emotional stress, or sepsis), lactate levels and/or trends may be mapped against other types of data, including the levels and/or trends of other analytes, as well as other non-analyte data, as shown at block 412. For example, to rule out lactate changes caused by the user eating a meal, the lactate levels and/or trends may be mapped against glucose levels and/or trends, the elevation and/or fluctuation of which may indicate consumption of a meal by the user. In another example, to rule out lactate changes caused by emotional stress or other confounding causes, the lactate levels and/or trends may be mapped against non-analyte sensor data from, e.g., a physical activity sensor such as heart rate monitor or accelerometer, which may confirm that the user is engaging in physical activity. Accordingly, lactate levels and/or lactate trends, in combination with glucose levels and/or trends and other analyte and/or non-analyte sensor data, may be utilized to determine whether the user is engaging in physical activity during the time period.

In certain embodiments, lactate measurements may further be utilized as a secondary filter to discriminate between glucose data sampling noise and actual glucose level acceleration. For example, since changes in lactate levels indicate physical stress, such as performance of physical activity and/or a change in intensity of physical activity, a change (e.g., rise) in lactate levels may confirm that a glucose spike is not sampling noise, but an actual glucose spike caused by physical stress, such as physical activity. Accordingly, the utilization of lactate may help increase the accuracy of predicting glycemic events by discriminating between sampling noise and actual changes in glucose level measurements.

In certain embodiments, performance of physical activity is determined utilizing data from a non-analyte sensor or device. For example, physical activity may be determined based on an elevated heart rate of the patient, or an increase in movement, and detected by non-analyte sensor(s), e.g. non-analyte sensor(s) 206. Other types of non-analyte sensor data that may be utilized to determine physical activity include accelerometer data, step rate data, exercise equipment power meter data, GPS data, heart rate data (e.g., heart rate reserve and HRV), EKG data, EMG data, respiration rate data, temperature data, blood pressure data, galvanic skin response data, oxygen uptake data, sleep data, impedance data, etc.

If decision support engine 114 determines that the user is engaging in physical activity during the time period, then at block 414, decision support engine 114 determines an intensity (or type) of the physical activity and a duration thereof. Determining the intensity of the physical activity and the duration thereof increases the accuracy of glycemic event prediction, and in particular, prediction of physical activity-induced glycemic events, since physical activity intensity may be indicative of a change in insulin sensitivity. For example, physical activity intensity may inform the determination of whether a patient is in an atypical state, which may impact, e.g., insulin sensitivity. Furthermore, the determination of whether the user is performing physical activity and/or the intensity thereof, when mapped to time, may further increase the accuracy of predicting overnight glycemic events. For example, performing physical activity in the evening (e.g., closer to the time a user normally goes to sleep), as compared to the morning or early afternoon (e.g., closer to the time a user normal wakes up), may lead to a greater likelihood of overnight glycemic events, such as hypoglycemia. Accordingly, determining when the user engages in physical activity may help inform a prediction of whether they will experience an overnight glycemic event.

In certain embodiments, upon determining the performance of physical activity by the user, decision support engine 114 may determine whether the user is engaging in low-to-moderate or moderate-to-high intensity physical activity.

During low-to-moderate intensity physical activity, increased cellular energy requirements are predominantly supplied by fat oxidation. The rates of lactate production and lactate clearance remain fairly balanced with one another, such that lactate levels remain relatively constant at or near a baseline concentration of the user. Glucose levels initially rise due to an immediate release of glucose from the liver, but eventually begin to diminish as glucose is transported intracellularly to supply energy for prolonged physical activity. This decline then steadily continues for the duration of the physical activity. During recovery from low-to-moderate intensity physical activity, and in particular, extended periods of low-to-moderate intensity physical activity (e.g., 30 minutes), glucose levels may continue to decline as in vivo insulin activity or delivered insulin may cause a decrease in glucose production, thereby leading to hypoglycemic events. Accordingly, extended periods of low-to-moderate intensity physical activity, may put a user at increased risk of hypoglycemia without some form of intervention or treatment. Low-to-moderate intensity physical activities include walking, light jogging, light cycling, light swimming, hiking, housework, gardening, and the like.

During moderate-to-high intensity physical activity, glucose is the primary provider of cellular energy, and glucose levels rise as glucose production is increased. In the absence of a sufficient supply of oxygen, increased glucose production and glycolysis results in increased production of lactate. With higher intensity physical activities, e.g., high intensity physical activity, the lactate production rate may surpass the rate of lactate clearance at the muscular level, thus causing a rise in lactate levels. At a certain point, lactate production may exceed lactate clearance, leading to a more pronounced increase in lactate levels. The intensity of physical activity at which lactate production exceeds lactate clearance and a pronounced increase in lactate levels occurs is referred to as the “lactate threshold,” which varies between users depending on their fitness level, exercise modality and/or other factors. Generally, a higher lactate threshold corresponds with greater fitness and endurance.

High intensity physical activity is associated with better glucose stability than low-to-moderate intensity physical activity. During recovery from high intensity physical activity, a user may experience high glucose levels, e.g., hyperglycemia, which may be referred to as a “glycemic rebound.” Such a glycemic rebound may occur as elevated lactate levels of the user normalize due to the user's body consuming the excess lactate as fuel. And while lactate is being utilized as the body's primary energy source, glucose levels may increase, eventually leading to a period of hyperglycemia. This glycemic rebound typically persist for about one hour. Glucose levels then equilibrate, with a reduced chance of, e.g., hypoglycemia thereafter, as compared to low-to-moderate intensity physical activity. In certain cases, engaging in high intensity physical activity before low-to-moderate intensity physical activity may lessen hypoglycemic effects of the low-to-moderate intensity physical activity, particularly for insulin-resistant diabetic patients. Examples of high intensity physical activity include sprinting, jumping rope, intense cycling, intense swimming, circuit training (HIIT), resistance training (weight lifting), and the like.

In certain embodiments, decision support engine 114 may determine the intensity of the physical activity based on lactate levels and/or trends alone. For example, upon determining that the user's lactate levels are relatively constant at or near a baseline concentration during the time period, decision support engine 114 may determine that the user is engaging in low-to-moderate intensity physical activity. In another example, upon determining that the user's lactate levels have exponentially increased and/or have exceeded the user's lactate threshold, decision support engine 114 may determine that the user is engaging in moderate-to-high or high intensity physical activity.

In certain embodiments, decision support engine 114 may determine the intensity of the physical activity based on lactate levels and/or trends in addition to analyte data and/or trends of one or more other analytes and/or other non-analyte data, as shown at block 416. For example, in certain embodiments, decision support engine 114 may determine the intensity of physical activity based on mappings of physical intensity to lactate levels and/or trends, glucose levels and/or trends, and non-analyte sensor or device data. For example, decision support engine 114 may access a reference library that has various lactate and glucose related ranges as well as ranges associated with non-analyte sensor or device data. Decision support engine 114 may then be configured with various rules to utilize the reference library to determine the intensity of the physical activity a user is or has engaged in based on the user's own data. For example, a simplified rule may state that “If the user's lactate levels are within X range, the user's glucose levels are within Y range, and the user's heart rate is within Z range, then the user is engaging in high intensity exercise.”

Examples of non-analyte sensor or device data include accelerometer data, step rate data, exercise equipment power meter data, GPS data, heart rate data (e.g., heart rate reserve and HRV), EKG data, EMG data, respiration rate data, temperature data, blood pressure data, galvanic skin response data, oxygen uptake data, sleep data, impedance data, etc., as provided by continuous or non-continuous non-analyte sensors described above. Other physiological parameters, such as heart rate, may be good indicators of physical activity intensity, depending on the type of physical activity. For example, heart rate may be a good indicator of aerobic, low intensity physical activities, such as walking and light jogging, aerobic, moderate intensity physical activities, such as swimming laps and cycling, as well as anaerobic, high intensity physical activities such as sprinting, jumping, and high intensity interval training (HIIT). Typically, low intensity physical activity results in the heart rate of the user hovering between about 40% and about 60% of a maximum heart rate of the user (e.g., 220—age of the user). Moderate intensity physical activity results in a heart rate of between about 50% and about 70% of the user's maximum heart rate. However, heart rate, as well as other non-analyte physiological parameters such as accelerometer data, may not be good indicators for certain anaerobic high-intensity physical activities, such as weight lifting. Therefore, using a combination of lactate, glucose, and non-analyte sensor or device data may provide a more accurate characterization of the intensity and/or type of physical activity engaged by the user.

At block 418, decision support system 100 generates a glycemic event prediction based on the determined physiological state of the patient, the analyte data for the plurality of analytes, the trend(s) of each of the plurality of analytes, and in certain embodiments, the other non-analyte sensor or device data. Examples of predicted glycemic events include hypoglycemia, hyperglycemia, increased insulin sensitivity, increased insulin resistance, as well as other metabolic disease events, such as hyperketonemia (DKA), hyperlactatemia, lactate acidosis, etc. Block 418 may be performed by decision support engine 114 illustrated in FIG. 1, in certain embodiments.

In certain embodiments, the glycemic event prediction generated by decision support system 100 is based, in part, on a determination of insulin sensitivity of the patient during or after the time period. As described above, insulin sensitivity may be informed by the determination of physical activity engaged by the patient, and/or the intensity thereof. For example, depending on the intensity and duration, physical activity may stimulate either short term (e.g., immediately upon physical activity and up to 72 hours thereafter) and long term (e.g., beyond 72 hours) changes, (increases or decreases) in insulin sensitivity. Thus, when factored together with the changes in analyte levels caused by the physical activity, insulin sensitivity may facilitate a more accurate prediction of physical activity-induced glycemic events.

In certain embodiments, changes in insulin sensitivity may be based on analyte levels and/or trends. For example, changes in insulin sensitivity may be determined based on measurements of glucose, ketones, glycerol, electrolytes such as sodium and potassium, calculated measurements such as anion gap, and other suitable analytes, including any of the analytes discussed herein. In certain embodiments, changes in insulin sensitivity may be based on lactate levels and/or trends. For example, if lactate levels of patient drop below the patient's lactate baseline after engaging in physical activity, this may indicate that the patient's insulin sensitivity is increased. Accordingly, after determining an increased insulin sensitivity, decision support system 100 may recommend adjusting/modifying insulin dosing until lactate levels of the patient return to baseline. For example, decision support system 100 may recommend that the patient adjust their insulin-to-carb ratio, e.g., reduce their insulin-to-carb ratio, to avoid hypoglycemia.

In certain embodiments, changes in insulin sensitivity may be based on lactate levels and/or trends, in addition to glucose levels and/or trends. For example, if a patient's lactate levels are decreasing after a post-physical activity lactate spike, and glucose levels of the patient are simultaneously increasing, this may indicate a glycemic rebound wherein the patient's insulin sensitivity may be reduced, since lactate is being used as the body's primary source of fuel. Accordingly, decision support system 100 may recommend to the patient to avoid dosing insulin, as doing so may result in insulin “stacking,” wherein insulin dosing causes increased insulin on board levels but does not immediately affect the patient's rising glucose levels since lactate is still being cleared.

In certain examples, decision support system 100 determines a change in insulin sensitivity of the patient for up to 12 hours, up to 24 hours, up to 36 hours, up to 48 hours, up to 60 hours, or up to 72 hours after the patient engaging in physical activity. Such change in insulin sensitivity may be determined and/or confirmed by, e.g., lower fasting lactate levels of the patient, and/or from an insulin pen or pump in communication with decision support system 100 and larger than normal responses of the patient to insulin administration. During this time period of insulin sensitivity, decision support system 100 may adjust one or more glucose thresholds of the user, as well as one or more parameters utilized in the determination of insulin dosing recommendations, meal recommendations, and/or other post-physical activity glycemic treatment recommendations. For example, in certain embodiments, such parameters may include desired insulin on board levels and/or insulin activity. Accordingly, in certain examples, the adjusted parameters are utilized to modify recommendations for insulin dosing. In certain examples, the adjusted parameters are utilized to modify meal recommendations.

In certain examples, decision support system 100 generates a prediction of hypoglycemia. Mild to severe hypoglycemia may occur as a result of decreased glucose levels caused by low-to-moderate intensity physical activity, in addition to increased insulin sensitivity and/or administered insulin. Hypoglycemia often occurs within about 45 minutes of prolonged low-to-moderate intensity physical activity, and may in certain cases arise at nighttime, particularly when physical activity is performed in the afternoon or evening. Hypoglycemic events can reduce the effectiveness of counter-regulatory responses in subsequent hypoglycemic events, as early as the next day. Thus, with consecutive days of low-to-moderate intensity physical activity, the likelihood and/or degree of induced hypoglycemia may increase, and predictions and/or recommendations thereof may be adjusted to account for this.

In certain examples, decision support system 100 generates a prediction of hyperglycemia. Mild to severe hyperglycemia may occur as a result of increased glucose levels caused by the liver's conversion of lactate to glucose during high intensity physical activity. During such high intensity physical activity, conversion of lactate to glucose by the liver may be resistant to increases in insulin. Following such high intensity physical activity, however, hyperglycemia may persist for approximately an hour before secretion of insulin stimulates glucose uptake and equilibrates glucose levels in the body.

Different methods for generating glycemic event prediction may be used by decision support engine 114. In certain embodiments, decision support engine 114 may use a rule-based model to provide real-time or retrospective decision support for physical activity-induced glycemic event prediction. Rule-based models involve using a set of rules for manipulating and/or analyzing data. These rules are sometimes referred to as “If Statements” as they tend to follow the line of “If X happens, then do Y.” In particular, decision support engine 114 may apply rule-statements (e.g., if, then statements) to assess the likelihood and/or occurrence of glycemic events.

For example, a first rule may be “If a patient's glucose levels are between X and Y, and the patient is engaging in low-to-moderate intensity physical activity for more than Z minutes, then the patient is likely to experience hypoglycemia within a certain amount of time,” while a second rule may be “If the glucose levels are between Y and Z, and the patient is engaging in high intensity activity, then the patient is likely to experience hyperglycemia within a certain amount of time.” The analyte data for the plurality of analytes, trend(s) of each of the plurality of analytes, and in certain embodiments, other non-analyte sensor or device data may be applied against these predefined rules to predict physical activity-induced glycemic events.

Such rules may be defined based on empirical research and maintained by decision support engine 114 in a reference library. For example, the reference library may maintain ranges of analyte levels, ranges of deltas in analyte trends, ranges of other non-analyte sensor and/or device data, etc. which may be mapped to different glycemic events. In certain embodiments, such rules may be determined based on training server system 140 analyzing historical patient records from historical records database 112.

In some cases, the reference library may become very granular. For example, other factors may be used in the reference library to create such “rules.” Other factors may include, gender, age, diet, disease history, physical fitness level, etc. Increased granularity may provide more accurate outputs. As an example, including fitness levels in the rule-based approach, e.g., used by decision support engine 114, may help inform differences in lactate thresholds or glucose levels such that glycemic event prediction by decision support engine 114 is more accurate. For example, a patient with greater physical fitness may have a higher lactate threshold as compared to the lactate threshold of a physically unfit patient and therefore, the patient with a higher level of fitness may be able to engage in longer periods of high intensity efforts without increasing the risk of physical activity-induced glycemic events, and/or is generally less likely to experience physical activity-induced glycemic events; thus, physical fitness may be an important factor to analyze in the rule-based approach to better predict physical activity-induced glycemic events.

In certain embodiments, as an alternative to using a rule-based model, AI models, such as machine learning models, may be used to provide real-time and retrospective decision support for physical activity-induced glycemic event prediction. In certain embodiments, decision support engine 114 may deploy one or more of these machine learning models for performing prediction of glycemic events of a user.

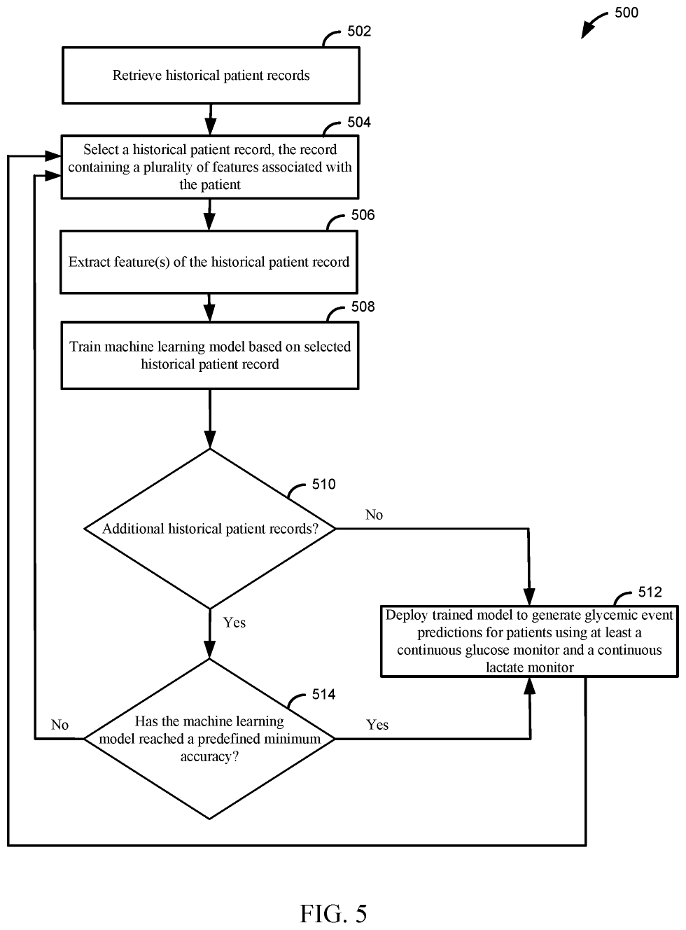

In particular, decision support engine 114 may obtain information from a user profile 118 associated with a user, stored in user database 110, featurize information for the user stored in user profile 118 into one or more features, and use these features as input into such models. Alternatively, information provided by the user's profile 118 may be featurized by another entity and the features may then be provided to decision support engine 114 to be used as input into the ML models. In machine learning, a feature is an individual measurable property or characteristic that is informative for analysis. In certain embodiments, features associated with the user may be used as input into one or more of the models to assess the likelihood and/or occurrence of glycemic events of the user. In certain embodiments, features associated with the user may be used as input into one or more of the models to identify glucose-stabilizing actions which may be provided as recommendations to the user for treatment of the predicted glycemic event, as described in block 420 below. Detail associated with how one or more machine learning models can be trained to provide real-time and retrospective decision support for glycemic event prediction are further discussed in relation to FIG. 5.

As previously mentioned, in certain embodiments, analyte data may be used by decision support engine 114 to generate a glycemic event prediction for a user, at block 418. Analyte data, including glucose and lactate data, as well as ketone data and/or any other analytes mentioned above (e.g., from measurements by continuous analyte monitoring system 104), may be used as input into such machine learning models and/or rule-based models to predict physical activity-induced glycemic events.

In some cases, method 400 continues at block 420 by decision support engine 114 generating one or more recommendations for treatment based, at least in part, on the glycemic event prediction at block 418. In particular, decision support engine 114 makes glycemic event treatment decisions or recommendations for the user. Treatment recommendations may include alarms and/or recommendations for immediate action, such as administration of a drug or consumption of food or modification of a physical activity, or recommendations for lifestyle modification. Examples of recommendations generated by decision support system 100 include: recommending administration of a small or regular insulin dose mid- or post-physical activity; recommending consumption of carbohydrates mid- or post-physical activity; recommending types of foods to consume or avoid post-physical activity; recommending a type, duration, and/or intensity of physical activity; recommending an active cool-down activity having reduced intensity (e.g., walking); recommending a mid-physical activity rest period; recommending an insulin dose or increased carbohydrate consumption prior to the next physical activity; recommending a therapy modification, etc. In certain embodiments, instead of a drug administration recommendation, decision support system 100 may calculate a recommended drug dosage and transmit the recommendation to a drug administration device (e.g., medical device 108) to automatically administer the recommended dosage of the drug to the user.

Decision support engine 114 may output such recommendations for treatment to the user (e.g., through application 106). In some embodiments, the recommendations may be displayed for viewing by the patient on, e.g., display device 107 illustrated in FIG. 1, and display devices 210, 220, 230, and 240 illustrated in FIG. 2.

In certain examples, where hypoglycemia is predicted, decision support system 100 may generate a recommendation for increased carbohydrate intake and/or a reduction in insulin dosage to maintain glucose stability, either during or after a current physical activity, or prior to engaging in the next physical activity, e.g., based on predicted behavior of the patient, including predicted physical activity and/or carbohydrate consumption. Aggressive reductions in insulin dosage, or a skipped dose, may lead to hyperglycemia before and during aerobic exercise and thus, a more moderate reduction in insulin dosage may be recommended.

In certain examples, where hyperglycemia is predicted, decision support system 100 may generate a recommendation for an additional dose of insulin following a physical activity, or for consumption of a meal prior to engaging in the next physical activity, e.g., based on predicted behavior of the patient. An additional, regular-sized dose of insulin may lead to hypoglycemia after physical activity and thus, instead, a smaller dose may be recommended. In certain examples, the decision support system 100 may recommend against taking short rest intervals between high intensity physical activities, as such may amplify hyperglycemia. Thus, longer rest intervals may be recommended instead. In certain embodiments, decision support system 100 may generate a recommendation for an additional dose of insulin during a physical activity.

In certain embodiments, where hyperlactatemia is determined and a state of increased insulin resistance is predicted due to the hyperlactatemia, decision support system 100 may generate a recommendation for an increase in insulin dosage until lactate levels normalize or return to baseline, e.g., based on predicted behavior of the patient.

In certain embodiments, after administration of an insulin dose, decision support system 100 may continue monitoring lactate levels of the user to determine a lactate response to the insulin dose, and thereafter adjust the predicted glycemic event based on the lactate response. For example, little to no change in lactate levels (e.g., less than 50% change in lactate levels) may indicate greater insulin sensitivity and thus, a more substantial change in glucose levels in response to the insulin dose. Alternatively, a large delta in lactate levels (e.g., a change of 50% or more in lactate levels), may indicate greater insulin resistance and thus, little to no change in glucose levels in response to the insulin dose.

In certain embodiments, where the user is determined to be engaging in physical activity during the time period at block 410, decision support system 100 may generate a recommendation to perform active recovery following the physical activity. Active recovery, or active cool-down, may comprise user performance of physical activity that gradually decreases in intensity to enable the user's body to gradually transition to a resting, or near-resting, state. Performance of active recovery following a physical activity may facilitate more rapid normalization of elevated lactate levels of the user as caused by the physical activity, which in turn, may lead to improved glycemic stability. For example, a user may exhibit elevated lactate levels after performance of a physical activity. At some time point following the physical activity, the user's lactate levels may begin to normalize (e.g., decrease) as the user's body consumes the excess lactate for energy. And while lactate is being utilized as fuel for the user's body, glucose levels of the user may begin to increase, since glucose is not being consumed, and insulin sensitivity of the user may decrease. The user's glucose may thus spike, and in certain embodiments, lead to hyperglycemia. By performing active recovery post-physical activity, however, the user may normalize their lactate levels faster, thus preventing or reducing the likelihood of transitioning into a hyperglycemic state and decreasing the patient's period of glucose insensitivity/resistance. Accordingly, decision support system 100 may in certain embodiments generate a recommendation to perform active recovery where such hyperglycemia is predicted. Examples of active recovery generally include jogging, walking, stretching, etc.

In embodiments where a recommendation for active recovery is generated, decision support system 100 may further provide guidance as to the type and duration of active recovery. In certain embodiments, decision support system 100 may provide guidance on how to achieve a desired lactate slope, or absolute lactate level reduction, so as to prevent a transition to a hyperglycemic state. In such embodiments, the guidance may be based on correlations of the user's historical lactate data (e.g., historical lactate levels and trends of the user) and the user's historical non-analyte sensor data, such as historical heart rate data and/or historical respiratory rate data or the user. In certain embodiments, the type and duration of active recovery recommended by decision support system 100 may further be based on a determination of a change in insulin sensitivity of the patient.

In certain embodiments, where decision support engine 114 determines the user is experiencing a glycemic rebound post-physical activity, decision support system 100 may generate a recommendation to avoid consumption of foods that would increase lactate levels and exacerbate the glycemic rebound (e.g., cause a further increase to the glucose spike).

In certain embodiments, where decision support engine 114 determines the user is engaging in physical activity during the time period at block 410, and/or decision support engine 114 determines the intensity, type, and/or duration of the physical activity at block 414, decision support system 100 may generate a recommendation to modify the intensity and/or type of the physical activity in order to facilitate improved glycemic stability post-physical activity. For example, during performance of short-duration, moderate-to-high intensity physical activity, the user may consume glucose at relatively fast rates but for a short time period, which can lead to a transient glycemic rebound after performing the physical activity with reduced risk of hypoglycemia. However, during performance of long-duration, low-to-moderate intensity physical activity, the user may consume glucose at relatively slow rates but for a long time period, which may not cause the same transient post-physical activity glycemic rebound as moderate-to-high intensity activity, thereby leading to increased risk of hypoglycemia. Accordingly, decision support system 100 may in certain embodiments, generate a recommendation to modify an intensity and/or type of physical activity where hypoglycemia (or hyperglycemia) is predicted. For example, where the user is performing long-duration, low-to-moderate intensity physical activity, decision support system 100 may generate a recommendation for the user to increase the intensity of the activity, e.g., towards the end of the time period, to facilitate a post-physical activity glycemic rebound.

FIG. 5 is a flow diagram depicting a method 500 for training machine learning models to provide a prediction of physical activity-induced glycemic events, according to certain embodiments of the present disclosure. In certain embodiments, the method 500 is used to train models to predict a current or future glycemic event in a patient engaging in physical activity, e.g., a user illustrated in FIG. 1.

Method 500 begins, at block 5602, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more patients who are not, or were not previously, users of continuous analyte monitoring system 104 and/or application 106.

Retrieval of data from historical records database 112 by training server system 140, at block 502, may include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, at block 502, training server system 140 may retrieve information for 100,000 patients with diabetes stored in historical records database 112 to train a model to predict a current or future physical activity-induced glycemic event in a user. Each of the 100,000 patients may have a corresponding data record, such as user profile 118 illustrated in FIG. 1, stored in historical records database 112. Each data record may include information, such as information discussed with respect to FIG. 3.

At block 504, method 500 continues by training server system 140 selecting one of the historical patient records retrieved by training server system 140 at block 502. The record contains information associated with the patient, such as the information stored in the patient's user profile. Examples of types of information included in a patient's user profile were provided above. Training server system 140 may use any suitable criteria (e.g., beginning with the oldest records, beginning with the most recent records, and the like) for selection of a historical patient record, as training server system 140 will iterate through each historical access record in the training set until all records have been used to train the machine learning model or the machine learning model is accurately predicting physical activity-induced glycemic events for each historical patient record input into the model.

At block 506, method 500 continues by training server system 140 extracting one or more features of the selected historical patient record. These features are extracted to be used as input features for the machine learning model(s). For example, a user profile associated with the patient selected at block 504 may include at least, information related to an age of the patient, a gender of the patient, a fitness level of the patient, an average change (e.g., average delta) in glucose levels for the patient during or after low to moderate intensity physical activity, an average change in glucose levels for the patient during or after moderate to high intensity physical activity, an average change in lactate levels for the patient during or after low to moderate intensity physical activity, an average change in lactate levels for the patient during or after moderate to high intensity physical activity, average time period between low to moderate intensity or moderate to high intensity physical activity and a glycemic event for the patient, etc. Features used to train the machine learning model(s) may vary in different embodiments.

At block 508, method 500 continues by training server system 140 training one or more machine learning models based on the selected historical patient record. In some embodiments, the training server does so by providing the features (e.g., extracted at block 506) as input into a model. This model may be a new model initialized with random weights and parameters, or may be partially or fully pre-trained (e.g., based on prior training rounds). Based on the input features, the model-in-training generates some output. The output may include predictions of a physical activity-induced glycemic events, previously recommended treatments, or similar metrics.

In certain embodiments, training server system 140 compares this generated output with the actual label associated with the historical patient record to compute a loss based on the difference between the actual result and the generated result. This loss is then used to refine one or more internal weights and parameters of the model (e.g., via backpropagation) such that the model learns to predict the occurrence of physical activity-induced glycemic events (or its recommended treatments) more accurately.

At block 510, method 500 continues by training server system 140 determining whether additional training is needed. This may include evaluating whether any additional historical patient records remain in the training data set. Where at block 510, training server system 140 determines all training data has been input into the machine learning model, at block 512, training server system 140 deploys the trained model(s) for physical activity-induced glycemic event prediction during runtime. In some embodiments, this includes transmitting some indication of the trained model(s) (e.g., a weights vector) that can be used to instantiate the model(s) on another device. For example, training server system 140 may deploy the trained model(s) to decision support engine 114. The models can then be used to assess, in real-time, the likelihood of a physical activity-induced glycemic event for a user using application 106.

Where at block 510, training server system 140 determines that not all historical patient records of the training data have been input into the model for training, at block 514, training server system 140 determines whether the model has reached a predefined minimum accuracy (e.g., 90% accuracy, 95% accuracy, etc.). Where the predefined minimum accuracy has not been met, training server system 140 determines additional training remains, and method 500 returns to block 504. Alternatively, where the machine learning model is predicting accurately the predefined minimum accuracy (e.g., 90% or 95% of the time predicting accurately), at block 512, training server system 140 deploys the trained model(s) for physical activity-induced glycemic event prediction during runtime.

By iteratively processing each data set corresponding to each historical patient, the model may be iteratively refined to generate accurate predictions of physical activity-induced glycemic event prediction for a patient.

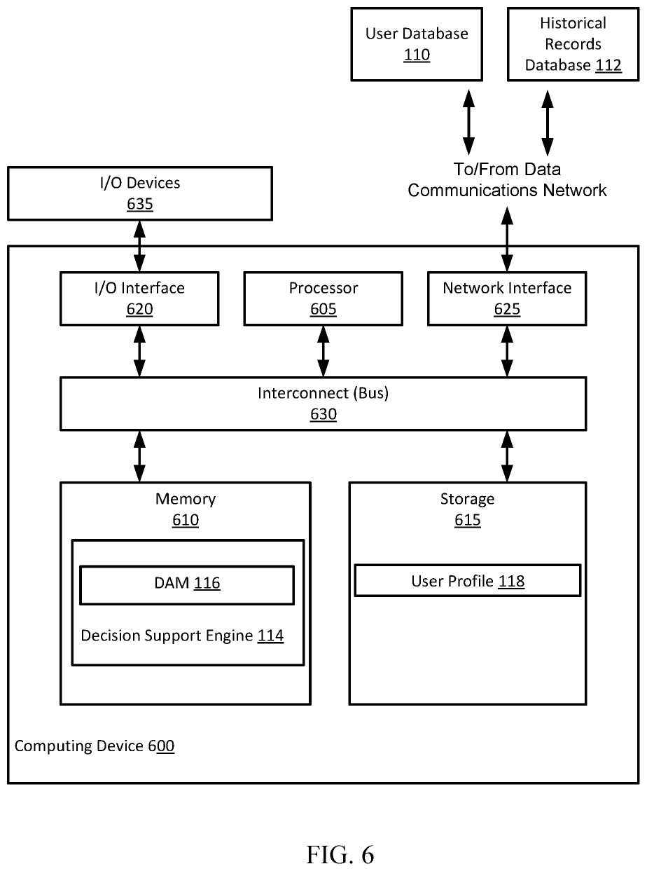

FIG. 6 is a block diagram depicting a computing device 600 configured for (1) predicting current or future physical activity-induced glycemic events, and/or (2) providing decision support for managing diabetes of patients as related to physical activity, according to certain embodiments disclosed herein. Although depicted as a single physical device, in embodiments, computing device 600 may be implemented using virtual device(s), and/or across a number of devices, such as in a cloud environment. As illustrated, computing device 600 includes a processor 605, memory 610, storage 615, a network interface 625, and one or more I/O interfaces 620. In the illustrated embodiment, processor 605 retrieves and executes programming instructions stored in memory 610, as well as stores and retrieves application data residing in storage 615. Processor 605 is generally representative of a single CPU and/or GPU, multiple CPUs and/or GPUs, a single CPU and/or GPU having multiple processing cores, and the like. Memory 610 is generally included to be representative of a random access memory (RAM). Storage 615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 635 (such as keyboards, monitors, etc.) can be connected via the I/O interface(s) 620. Further, via network interface 625, computing device 600 can be communicatively coupled with one or more other devices and components, such as user database 110 and/or historical records database 112. In certain embodiments, computing device 600 is communicatively coupled with other devices via a network, which may include the Internet, local network(s), and the like. The network may include wired connections, wireless connections, or a combination of wired and wireless connections. As illustrated, processor 605, memory 610, storage 615, network interface(s) 625, and I/O interface(s) 620 are communicatively coupled by one or more interconnects 630. In certain embodiments, computing device 600 is representative of display device 107 associated with the user. In certain embodiments, as discussed above, display device 107 can include the user's laptop, computer, smartphone, and the like. In another embodiment, computing device 600 is a server executing in a cloud environment.

In the illustrated embodiment, storage 615 includes user profile 118. Memory 610 includes decision support engine 114, which itself includes DAM 116. Decision support engine 114 is executed by computing device 600 to perform operations in workflow 400 of FIGS. 4A-4B and/or operations of method 500 in FIG. 5 for predicting current or future physical activity-induced glycemic events, and/or providing decision support for managing diabetes of patients as related to physical activity.

### Example Embodiments

Embodiment 1: In certain embodiments, a method for generating a glycemic event prediction is provided, the method comprising: continuously monitoring a plurality of analytes of a patient during a time period to obtain analyte data; processing the analyte data from the time period to determine a trend of each of the plurality of analytes; determining a physiological state of the patient based on the trend of each of the plurality of analytes, wherein determining the physiological state of the patient comprises determining whether the patient is engaging in physical activity; and predicting a current or future glycemic event of the patient based on the physiological state of the patient, the analyte data, and the trend of each of the plurality of analytes.

Embodiment 2: The method of Embodiment 1, wherein the plurality of analytes comprises at least lactate and glucose.

Embodiment 3: The method of Embodiment 1, wherein determining the physiological state of the patient further comprises determining an intensity level of the physical activity engaged by the patient.

Embodiment 4: The method of Embodiment 1, wherein determining whether the patient is engaging in physical activity is based on the analyte data and/or trends of the plurality of analytes.

Embodiment 5: The method of Embodiment 1, further comprising: generating one or more recommendations for treatment for the patient based, at least in part, on the current or future glycemic event of the patient.

Embodiment 6: The method of Embodiment 5, wherein the one or more recommendations for treatment comprise at least one of: a drug administration recommendation; a therapy modification recommendation; a food consumption recommendation; or a physical activity modification recommendation.

Embodiment 7: The method of Embodiment 1, wherein the plurality of analytes further include at least one of ketones, glycerol, potassium, and sodium.

Embodiment 8: The method of Embodiment 1, further comprising: monitoring other sensor data of the patient during the time period using one or more other non-analyte sensors.

Embodiment 9: The method of Embodiment 8, wherein the one or more other non-analyte sensors comprise at least one of an accelerometer, an impedance sensor, an electrocardiogram (EKG) sensor, a blood pressure sensor, a heart rate monitor, or a respiratory sensor.

Embodiment 10: The method of Embodiment 1, wherein the analyte data for lactate is utilized to discriminate between sampling noise and actual analyte data for glucose.

Embodiment 11: The method of Embodiment 1, wherein the glycemic event prediction is generated using a model trained using training data to predict a glycemic event induced by physical activity of the patient.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A method for generating a glycemic event prediction, the method comprising:
continuously monitoring a plurality of analytes of a patient during a time period to obtain analyte data, the plurality of analytes including at least lactate and glucose;
processing the analyte data from the time period to determine a trend of each of the plurality of analytes;
determining a physiological state of the patient based on the trend of each of the plurality of analytes, wherein determining the physiological state of the patient comprises determining whether the patient is engaging in physical activity; and
predicting a current or future glycemic event of the patient based on the physiological state of the patient, the analyte data, and the trend of each of the plurality of analytes.

2. The method of claim 1, wherein determining the physiological state of the patient further comprises determining an intensity level of the physical activity engaged by the patient.

3. The method of claim 1, wherein determining whether the patient is engaging in physical activity is based on the analyte data and/or trends of the plurality of analytes.

4. The method of claim 1, further comprising:
generating one or more recommendations for treatment for the patient based, at least in part, on the current or future glycemic event of the patient.

5. The method of claim 4, wherein the one or more recommendations for treatment comprise at least one of:
a drug administration recommendation;
a therapy modification recommendation;
a food consumption recommendation; or
a physical activity modification recommendation.

6. The method of claim 1, wherein the plurality of analytes further include at least one of ketones, glycerol, potassium, and sodium.

7. The method of claim 1, further comprising:
monitoring other sensor data of the patient during the time period using one or more other non-analyte sensors.

8. The method of claim 7, wherein the one or more other non-analyte sensors comprise at least one of an accelerometer, an impedance sensor, an electrocardiogram (EKG) sensor, a blood pressure sensor, a heart rate monitor, or a respiratory sensor.

9. The method of claim 1, wherein the analyte data for lactate is utilized to discriminate between sampling noise and actual analyte data for glucose.

10. The method of claim 1, wherein the glycemic event prediction is generated using a model trained using training data to predict a glycemic event induced by physical activity of the patient.

11. A system for providing glycemic event decision support, the system comprising:
one or more continuous analyte sensors, the one or more continuous analyte sensors configured to continuously monitor a plurality of analytes of a patient during a time period to obtain analyte data, the plurality of analytes including at least lactate and glucose; and
one or more memories comprising executable instructions;
one or more processors in data communication with the one or more memories and configured to execute the instructions to:
process the analyte data from the time period to determine a trend of each of the plurality of analytes;
determine a physiological state of the patient based on the trend of each of the plurality of analytes, wherein determining the physiological state of the patient comprises determining whether the patient is engaging in physical activity; and
predict a current or future glycemic event of the patient based on the physiological state of the patient, the analyte data, and the trend of each of the plurality of analytes.

12. The system of claim 11, wherein determining the physiological state of the patient further comprises determining an intensity level of the physical activity engaged by the patient.

13. The system of claim 11, wherein determining whether the patient is engaging in physical activity is based on the analyte data and/or trends of the plurality of analytes.

14. The system of claim 1, wherein the one or more processors are further configured to:
generate one or more recommendations for treatment for the patient based, at least in part, on the current or future glycemic event of the patient.

15. The system of claim 14, wherein the one or more recommendations for treatment comprise at least one of:
a drug administration recommendation;
a therapy modification recommendation;
a food consumption recommendation; or
a physical activity modification recommendation.

16. The system of claim 15, wherein the plurality of analytes further include at least one of ketones, glycerol, potassium, and sodium.

17. The system of claim 11, further comprising:
one or more non-analyte sensors, the one or more non-analyte sensors configured to monitor non-analyte sensor data of the patient during the time period, wherein the one or more processors are further configured to process the non-analyte sensor data from the time period and determine the physiological state of the patient based on the processed non-analyte sensor data.

18. The system of claim 17, wherein the one or more non-analyte sensors comprise at least one of an accelerometer, an impedance sensor, an electrocardiogram (EKG) sensor, a blood pressure sensor, a heart rate monitor, or a respiratory sensor.

19. The system of claim 11, wherein the one or more processors are further configured to utilize the analyte data for lactate to discriminate between sampling noise and actual analyte data for glucose.

20. The system of claim 11, wherein the one or more processors are further configured to generate the glycemic event prediction using a model trained using training data to predict a glycemic event induced by physical activity of the patient.
